(19) United States
(12) Patent Application Publication
(10) Pub. No.: US 2026/0338760 A1
(43) Pub. Date: Oct. 29, 2026

(54) METHOD AND KIT FOR REREADING SINGLE PROTEIN MOLECULES USING A NANOPORE AND AN UNFOLDASE

(71) Applicant: Oxford Nanopore Technologies PLC, Oxford (GB)

(72) Inventors: Jeffrey Matthew Nivala, Seattle, WA (US); Keisuke Motone, Seattle, WA (US)

(73) Assignee: Oxford Nanopore Technologies PLC, Oxford (GB)

(21) Appl. No.: 19/486,433

(22) PCT Filed: May 17, 2024
(86) PCT No.: PCT/US2024/030071

§ 371 Date: Nov. 21, 2025

Related U.S. Application Data
(60) Provisional application No. 63/470,301, filed on Jun. 1, 2023.
(60) Provisional application No. 63/542,193, filed on Oct. 3, 2023.

(51) Int. Cl. G01N 33/487; C12Q 1/34

(52) U.S. Cl. CPC G01N 33/48721; C12Q 1/34; C12Y 306/0401

(57) ABSTRACT

Methods and kits for analyzing a polypeptide are described. In an embodiment, the method comprises providing, into a first compartment of a nanopore sequencing device, one or more polypeptides comprising: a polyanion sequence; a folded domain stably folded at neutral pH and/or a positively charged sequence; an unfoldase slip sequence; and an analyte sequence, applying an electrophoretic and/or electroosmotic force between the first compartment and a second compartment of the nanopore sequencing device to translocate partially the one or more polypeptides from the first compartment to the second compartment through a nanopore of the nanopore sequencing device; translocating, with an unfoldase enzyme, the one or more polypeptides partially through the nanopore from the second compartment to the first compartment; and measuring a current corresponding to moving, with the unfoldase enzyme, the one or more polypeptides from the second compartment to the first compartment.

### RELATED APPLICATIONS

This application is a national stage filing under 35 U.S.C. § 371 of international PCT application PCT/US2024/030071, filed May 17, 2024, which claims priority under 35 U.S.C. § 119 (e) to U.S. provisional patent application, U.S. Ser. No. 63/542,193, filed Oct. 3, 2023 and which claims priority under 35 U.S.C. § 119 (e) to U.S. provisional patent application, U.S. Ser. No. 63/470,301, filed Jun. 1, 2023, the entire contents of each of which are incorporated by reference herein.

### STATEMENT OF GOVERNMENT LICENSE RIGHTS

This invention was made with government support under Grant No. 1R01HG012545, awarded by the National Institutes of Health. The government has certain rights in the invention.

### REFERENCE TO AN ELECTRONIC SEQUENCE LISTING

The contents of the electronic sequence listing (0036670169US00-SEQ-KZM.xml; Size: 13,987 bytes; and Date of Creation: Nov. 3, 2025) are herein incorporated by reference in their entirety.

### BACKGROUND

Annotating the complexity of protein variation is important in understanding biological processes, identifying disease states, and developing effective therapeutics. Proteoform diversity is a term that refers to the vast array of protein variations that can exist due to differences in transcription, translation, and post-translational modifications (PTMs) which can occur through enzymatic (e.g., phosphorylation) and non-enzymatic (e.g., spontaneous deamidation) processes. These variations occur independently and in combination with each other on single protein molecules, creating a “PTM code” that plays unique and specific roles in driving biological processes. The ability to sequence single protein molecules in their natural, full-length state would be valuable for better understanding this proteoform diversity and its underlying code. However, certain conventional protein sequencing and fingerprinting methods, including Edman degradation and mass spectrometry, have difficulty analyzing full-length proteins from complex samples and face challenges with detection sensitivity, dynamic range, analytical throughput, and instrumentation cost. To address these challenges, complementary or potentially disruptive platforms for next-generation protein analysis and sequencing have been proposed, including single-molecule fluorescence labeling and affinity-based approaches. However, these emerging techniques also face limitations compared to nanopore technology, which has the potential to achieve direct, label-free, full-length protein sequencing.

Nanopore technology uses a nanometer-sized pore within an insulating membrane that separates two electrolyte-filled wells. A voltage applied across the membrane drives ionic current flow through the nanopore sensor. When individual analyte molecules pass through the pore, they can manifest a detectable signal change. This change can provide insight into the molecular nature of the analyte. Although originally envisioned and now commercialized as a technique for sequencing nucleic acid strands, nanopore sensing holds great potential for protein analysis. It has been used for discrimination of peptides and proteins, real-time measurement of protein-protein, and protein-ligand interactions, and aptamer-mediated protein detection. Additionally, protein nanopores have shown promise in identifying amino acids and PTMs, such as phosphorylation and glycosylation, which serve as important biomarkers of cell states and diseases. Recent work has demonstrated some ability to read DNA-conjugated peptide strands using DNA-processive molecular motors, such as a helicase or polymerase. Further, rereading of peptide fragments using this strategy made it possible to resolve amongst a small subset of single-amino acid substitutions with high accuracy.

Despite this progress, obtaining sequence information from intact, full-length proteins using nanopores has been hindered by the difficulty in driving long protein strands through the sensor, which arises due to the neutrally charged polypeptide backbone, varying charge states of amino acid side chains, and stable tertiary structures.

### SUMMARY

To overcome the challenges of reading full-length protein molecules, the present disclosure provides a technique to reversibly thread long protein strands into a nanopore, such as a CsgG pore, by electrophoresis, and then enzymatically pull them back out of the pore using the protein unfoldase/translocase activity of an unfoldase protein, such as ClpX. While the initial stage of threading the protein into the pore using electrophoretic force happens too quickly to resolve any sequence attributes, unfoldase-mediated translocation of proteins back out of the pore manifests slow, reproducible ionic current signals. This method enabled the processive translocation of long proteins, facilitating the detection of numerous single amino acid substitutions and PTMs across protein strands. The present disclosure also provides an approach to rereading the same protein strand multiple times. Furthermore, the present disclosure demonstrates the methods of the present disclosure enable unfolding and translocation of entire folded protein domains for linear, end-to-end analysis.

Accordingly, in an aspect the present disclosure provides a method of analyzing a polypeptide. In an embodiment, the method comprises providing, into a first compartment of a nanopore sequencing device, one or more polypeptides comprising a polyanion sequence; a folded domain stably folded at neutral pH and/or a positively charged sequence; an unfoldase slip sequence; and an analyte sequence, applying an electrophoretic and/or electroosmotic force between the first compartment and a second compartment of the nanopore sequencing device to translocate partially the one or more polypeptides from the first compartment to the second compartment through a nanopore of the nanopore sequencing device; translocating, with an unfoldase enzyme, the one or more polypeptides partially through the nanopore from the second compartment to the first compartment; and measuring a current corresponding to moving, with the unfoldase enzyme, the one or more polypeptides from the second compartment to the first compartment.

In an embodiment, the unfoldase slip sequence is configured to cause the one or more polypeptides to translocate from the first compartment into the second compartment.

In an embodiment, a current pattern from the measured current corresponds to translocating, with the unfoldase, the one or more polypeptides at least partially from the second compartment to the first compartment a plurality of times.

In an embodiment, the method further comprises combining portions of the current pattern corresponding to translocating the one or more polypeptides from the second compartment to the first compartment to provide a combined current pattern. In an embodiment, the method further comprises comparing a portion of the combined current pattern corresponding to the analyte sequence to a reference current pattern obtained using a reference polypeptide measured under analogous conditions. In an embodiment, the method further comprises generating an analyte sequence call based on comparing the portion of the combined current pattern corresponding to the analyte sequence to reference current pattern the reference polypeptide analyte measured under analogous conditions.

In an embodiment, the folded domain is disposed in a C-terminal direction relative to the analyte sequence.

In an embodiment, the unfoldase slip sequence is disposed in an N-terminal direction relative to the analyte sequence.

In an embodiment, the positively charged sequence is disposed in a C-terminal direction relative to the analyte sequence.

In an embodiment, the positively charged sequence is disposed in an N-terminal direction relative to the analyte sequence.

In an embodiment, the polyanion sequence is disposed in a C-terminal direction relative to the analyte sequence.

In an embodiment, the unfoldase slip sequence comprises polyproline sequences.

In an embodiment, the unfoldase slip sequence comprises one or more of a sequence according to SEQ ID NO. 1.

In an embodiment, the unfoldase slip sequence comprises one or more of a sequence according to SEQ ID NO. 2.

In an embodiment, the unfoldase comprises an amino acid sequence at least 90% or 95% identical to the amino acid sequence of SEQ ID NO: 3.

In an embodiment, the nanopore comprises an amino acid sequence at least 90% or 95% identical to the amino acid sequence of SEQ ID NO: 4.

In an embodiment, the polyanion sequence comprises a plurality of amino acid residues that are anionic under neutral pH.

In an embodiment, the polyanion sequence comprises at least 10 amino acid residues, at least 20 amino acid residues, at least 30 amino acid residues, at least 40 amino acid residues, or at least 50 amino acid residues.

In an embodiment, the polyanion sequence is configured to facilitate voltage-mediated capture of the one or more polypeptides in the nanopore.

In an embodiment, the polyanion sequence comprises amino acids selected from the group consisting of aspartic acid and glutamic acid.

In an embodiment, the folded domain is configured to prevent complete translocation of the one or more polypeptides through the nanopore.

In an embodiment, the folded domain comprises a tertiary structure larger than a vestibule of the nanopore.

In an embodiment, positively charged sequence prevents complete translocation of the one or more polypeptide through the nanopore.

In an embodiment, positively charged sequence comprises amino acid residues selected from the group consisting of arginine, histidine, and lysine.

In an embodiment, the unfoldase is configured to unfold the folded domain.

In an embodiment, the one or more polypeptides further comprise an ssrA tag.

In an embodiment, the ssRA comprises a sequence according to SEQ ID NO: 5.

In an embodiment, the ssRA is disposed in a C-terminal direction of the analyte sequence.

In an embodiment, the method further comprises modifying one or more sample polypeptides to comprise the polyanion sequence; the folded domain, the positively charged sequence; and the unfoldase slip sequence to provide the one or more polypeptides.

In another aspect, the present disclosure provides a kit. In an embodiment, the kit comprises a nanopore sequencing device comprising a first compartment; a second compartment; a nanopore disposed in an insulating membrane fluidically separating the first compartment from the second compartment; an unfoldase disposed in either the first compartment or the second compartment; and one or more unfoldase slip sequences.

In an embodiment, the kit comprises reagents configured to functionalize a sample polypeptide with the one or more unfoldase slip sequences.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this present disclosure will become more readily appreciated as the same become better understood by reference to the following detailed description, when taken in conjunction with the accompanying drawings, wherein:

FIGS. 1A-1C. Nanopore protein reading using an unfoldase. (1A) Schematic of the unfoldase approach on the nanopore sequencing device. Assigned Roman numerals correspond to ionic current states in 1B; (1B) Example trace of protein P1. Deep spikes within the capture state are hypothesized to be transient structural fluctuations of the Smt3 domain within the pore. State iii can be discerned from a transient drop in current when the ClpX solution is initially loaded into the flow cell. (1C) Ensemble traces of protein P1 (n=34) and mutants P2 (n=17), P3 (n=21), and P4 (n=12). Protein sequences are oriented from C-to-N.

FIGS. 2A-2I: Detecting single amino acid mutations across long protein strands. (2A) PASTOR sequence composition. (2B) Filtered nanopore current trace of a PASTOR according to embodiments of the present disclosure. Regions' boundaries are defined by automated YY-segmentation. (2C) Average signal trace for each amino acid's transformed VRs, after Euclidian alignment of all the VRs equidistantly stretched to the same length. VRs corresponding to a charged amino acid are shown in a dashed line. (2D) Scatter plot of various features of the uncharged VRs, with error bars denoting one standard deviation and explanation of the features to the right. n varies from 56 to 98. (2E) Bar blot of the variance of the max value of the transformed VRs corresponding to each amino acid. (2F) t-SNE map showing clustering of the pairwise DTW distance between each amino acid (2G) Plot of all the VRs corresponding to asparagine in normal conditions (left) and in conditions that catalyze the deamidation of asparagine to aspartate (right). n=81 for normal conditions and 77 for deamidation conditions. (2H) Bar plot displaying percent of mutations that have been putatively deamidated or not (same threshold as in 2G and 2H) in VRs corresponding to asparagine, across technical replicates with n=6, 4, 3, and 3 from left to right. Error bars denote standard deviations. (2I) t-SNE plot as in f, showing only asparagine and aspartate VRs. Asparagine VRs form a distinct cluster from aspartate and putative deamidated asparagine VRs (p_{PERMANOVA}<1×10⁻⁶). Putative deamidated asparagine and aspartate are indistinguishable (p_{PERMANOVAa}=0.8).

FIGS. 3A-3D: ClpX stepping behavior. (3A) shows an example PASTOR trace and bottom panels show its manually segmented YY dips. Black horizontal lines denote individual steps. (3B) Number of steps for each of the YY dips without back steps, n=776. (3C) Distribution of the mean number of residues per step within each of the YY dips. (3D) Step dwell time distribution.

FIGS. 4A-4E: Single-molecule nanopore sequencing of single amino acid mutations. (4A) PASTOR VR classification pipeline. (4B) Heatmap showing test accuracies in discriminating between all pairs of amino acid VR mutations, averaged over five Random Forests. (4C) Accuracy in a 20-way classification when “accuracy” is defined as the correct label being in the top-N most probable classes. The dummy classifier chooses one label at random. Results averaged over 20 models. (4D) and (4E) Example sequencing traces in the test set, for two PASTOR constructs HDKER and AVLIM. Transformed ionic current traces are plotted with a box around the variable regions defined by the segmentor. The intensity of the boxes represents the ranking of the true class in the aminocaller's prediction for each VR. For the 5-way classification task (top box shading), the classes are the 5 mutations found in that protein, while the 20-way classification task (bottom box shading) considers all possible amino acid classes. In each box, the letter corresponds to the model's top prediction, with the top letter denoting the 5-way classification and the bottom letter indicating the 20-way classification. A darker shade implies a more accurate prediction, indicating that the correct label ranked high in the model's predictions.

FIGS. 5A-5G: Rereading single protein molecules multiple times with an unfoldase slip sequence. (5A) Working model of rereading. The ClpX motor translocates PASTOR-reread to generate Read 1. ClpX releases the protein strand upon reaching the slip sequence, and electrophoresis drives back-slipping (first compartment to second compartment). ClpX regains grip and ratchets the protein strand through the nanopore again. Recurrent back-slipping events produce subsequent rereads. (5B) Top box: Example trace of PASTOR-reread showing three near complete reread events. Our model's predicted signal for the PASTOR-reread sequence was aligned to each reread. The fourth VR contains an asparagine mutation, but the corresponding signal level consistently resembles aspartate in all three instances for this particular PASTOR-reread trace. The modeled sequence was changed to contain an aspartate to reflect the putative PTM. These rereading data provide additional evidence supporting the notion that the variability observed in asparagine VRs is attributed to post-translational deamidation of N to (iso) D, and not read-to-read variation. Bottom box: plot showing the approximate region of the strand that is within the nanopore over time. (5C) Estimated back-slipping distance for ClpX concentrations at 1000 nM (n=141), 200 nM (n=609), 40 nM (n=777), and 8 nM (n=999). The very first full-length read (Read 1) of each analyte protein molecule was excluded from this analysis. (5D) and (5E) Number of all reads and full-length reads per PASTOR-reread molecule, respectively. The dotted lines indicate medians for ClpX concentrations at 1000 nM (n=26), 200 nM (n=37), 40 nM (n=23), and 8 nM (n=20). (5F) Simulated effect of rereading on 2 (Y, D), 4 (A, W, R, D), 7 (G, Q, W, F, R, D, E), 10 (A, G, V, N, Y, W, F, R, D, E), 14 (C, A, G, T, V, N, Q, M, Y, W, F, R, D, E), 17 (C, S, A, G, T, V, N, Q, M, I, Y, W, F, H, R, D, E), and 20-way (all 20 a.a.) classification tasks, compared to a baseline random classifier. Each value is the average over 100 train-test trials. (5G) Projected sequencing accuracy of barcode designs using the accuracies from 5F;

FIGS. 6A-6E: Single-molecule mapping kinase phosphorylation activity. (6A) Transformed current traces of PASTOR-phos, where each section—C-terminal linker, VR V, VR GLSARRL (SEQ ID NO: 11), VR A, and N-terminal linker—are aligned to the lowest DTW-distance phosphorylation state model of the corresponding section. Each of the four YY dips are aligned to the model of a YY dip (denoted by light beige rectangles). For PKA incubation, blank incubation (negative control), 1 hr CKII incubation, and 26 hr CKII incubation, n of traces=92, 155, 16, 171, respectively and n of experiments=2, 2, 1, 3, respectively. (6B) Ensemble traces of VR GLSARRL (SEQ ID NO: 11) for each of the four conditions. (6C) Maximum transformed signal value for each trace. Transparency of each scatter point is proportional to the n of traces for that condition. For each of C-terminal linker, VR V, VR A, and N-terminal linker conditions, the CKII incubation conditions' maximum values were significantly higher than the blank and PKA incubation conditions (p_{Mann-Whitney, one-sided}<10⁻⁸ for each, after Bonferroni correction). GLSARRL (SEQ ID NO: 11) region corresponds to the C-terminal third of the VR. The GLSARRL (SEQ ID NO: 11) region's maximum values are significantly higher in the PKA than the blank incubation condition (p_{Mann-Whitney, one-way}=5×10⁻³⁹, after Bonferroni correction) and the two CKII incubation conditions are significantly different from each of the two other conditions (p_{Mann-Whitney} <10⁻⁵ for each, after Bonferroni correction). (6D) Interquartile range (IQR) of the number of putative phosphorylations per molecule. No kinase incubation shows fewer phosphorylations than 1 hr incubation (p_{Mann-Whitney, one-way}=4×10⁻¹⁶), and 1 hr incubation in CKII shows fewer phosphorylations than 26 hr incubation (p_{Mann-Whitney, one-way}=5×10⁻⁶). Center line, box, whiskers, and diamonds represent median, IQR, 1.5 IQR, and outliners, respectively. (6E) Frequency of molecules best matching each proteoform, normalized by total counts for each experimental condition. CKII incubation conditions are stacked. Numbers about the bars show the number of phosphorylations contained within the proteoform (Proteoform ID1 contains no phosphorylations). * P<10⁻⁵; and

FIGS. 7A-7G: Processive reading of folded protein domains. (7A) Working model of ClpX-mediated processing of folded proteins. Assigned Roman numerals correspond to ionic current states in FIGS. 7B, 7C, and 7E. (7B), Example trace of PASTOR-Titin. (7C), Example trace of PASTOR-dTitin. (7D), Ensemble traces of state vii of PASTOR-Aβ15 (n=21), -Aβ42 (n=15), -Titin (n=20), and -dTitin (n=12). Protein sequences are shown in the C-to-N direction, and asterisks represent the C47E and C63E mutations between Titin and dTitin. (7E), Example trace of PASTOR-Aβ42. f, t-SNE plot based on pairwise DTW distances for state vii, showing Aβ15 and Aβ42 form a distinct cluster from Titin and dTitin (p_{PERMANOVA}≤1×10⁻⁶). Aβ15 vs Aβ42 and Titin vs dTitin states vii are indistinguishable (p_{PERMANOVA}=0.99, 0.67, respectively). (7G), Relationship between protein length and translocation time. State vii dwell time is plotted for Aβ15, Aβ42, Titin, and dTitin, as well as translocation time for the 8 PASTORs with no folded domain insert (n=672). The dotted line was fitted with the mean dwell times of each protein class (slope corresponds to a translocation rate of 16 ms/aa or 63 aa/sec, R²=0.998).

### DETAILED DESCRIPTION

The ability to sequence single protein molecules in their native, full-length form would enable a more comprehensive understanding of proteomic diversity. Current technologies, however, are limited in achieving this goal. Here, the present disclosure provides, in various aspects, a method for long-range, single-molecule reading of intact protein strands on a commercial nanopore sensor array. By using an unfoldase to ratchet proteins through a nanopore, the present disclosure provides evidence that the unfoldase translocates substrates in two-residue steps. This mechanism achieves single-amino acid level sensitivity, enabling sequencing of combinations of amino acid substitutions and mapping of post-translational modifications such as phosphorylation across long protein strands. To enhance sequencing accuracy further, the present disclosure demonstrates the ability to reread individual protein molecules, spanning hundreds of amino acids in length, multiple times, and explore the potential for high accuracy protein barcode sequencing. Further, the present disclosure provides a biophysical model that can simulate raw nanopore signals a priori, based on residue volume and charge, enhancing the interpretation of raw signal data. Additionally, the methods of the present disclosure are applied to examine intact, folded protein domains for complete end-to-end analysis. These results provide proof-of-concept for a platform that has the potential to identify and characterize full-length proteoforms at single-molecule resolution.

As discussed further herein, in an embodiment, an unfoldase enzyme (e.g., ClpX) is used to pull (translocate) nanopore-captured substrate proteins back out of the nanopore sensor. Substrate proteins are tagged at the N-terminal with a polyanion that facilitates their voltage-mediated capture in the nanopore. A folded domain and/or positively charged polypeptide region at the C-terminal prevents complete translocation of the substrate protein through the pore. Following capture of the substrate protein in the pore, an unfoldase motor can be loaded into the nanopore flow cell. The unfoldase (e.g., ClpX) then binds to a C-terminal tag present on the substrate protein (e.g., ssrA tag), and pulls the substrate protein back out of the pore. This method enables amino acid sequence dependent features of the substrate protein to be analyzed as it translocated through the pore by the unfoldase motor.

Accordingly, in an aspect, the present disclosure provides a method of analyzing a polypeptide, such as to provide a sequence call based on a primary sequence of the polypeptide.

In an embodiment, the method comprises providing, into a first compartment of a nanopore sequencing device, one or more polypeptides. In an embodiment, the one or more polypeptides comprise polypeptides from a sample, such as a biological sample comprising analyte sequences for sequencing.

In an embodiment, the one or more polypeptides comprise sequences, in addition to any analyte sequences, which aid in translocating the one or more polypeptides through a nanopore. Accordingly, in an embodiment, the one or more polypeptides comprise a polyanion sequence; a folded domain stably folded at neutral pH and/or a positively charged sequence; an unfoldase slip sequence; and an analyte sequence.

As above, in an embodiment, the one or more polypeptides comprise a polyanion sequence. In an embodiment, the polyanion sequence comprises a plurality of amino acid residues, such as a plurality of residues that are anionic under neutral pH. Without wishing to be bound by any particular theory, it is believed that the polyanion sequence is configured to facilitate voltage-mediated capture of the one or more polypeptides in the nanopore.

In an embodiment the polyanion sequence comprises a length sufficient to facilitate such voltage-mediated capture, such as by comprising a length configured to electrostatically or otherwise be attracted to an interior portion, such as a vestibule, of a nanopore. Accordingly, in an embodiment, the polyanion sequence comprises at least 10 amino acid residues, at least 20 amino acid residues, at least 30 amino acid residues, at least 40 amino acid residues, or at least 50 amino acid residues, or more.

As above, the polyanion sequence comprises amino acid residues that are anionic, such as at neutral pH or under other reaction conditions. Accordingly, in an embodiment, the polyanion sequence comprises amino acids selected from the group consisting of aspartic acid and glutamic acid.

As above, in an embodiment, the one or more polypeptides comprise a folded domain stably folded at neutral pH. In an embodiment, the folded domain is configured to prevent complete translocation of the one or more polypeptides through the nanopore. As discussed further herein, in this regard, the one or more polypeptides are configured to repeatedly pass through the nanopore, such as for repeated measurements of electrical current through the nanopore during such repeated translocation.

In an embodiment, the folded domain comprises a tertiary structure larger than a vestibule of the nanopore, such as configured to prevent complete translocation of the one or more polypeptides through the nanopore.

As above, in an embodiment, the one or more polypeptides comprises a positively charged sequence, such as comprising positively charged amino acid residues under neutral pH or other reaction conditions. Without wishing to be bound by any particular theory, it is believed that the positively charged sequence prevents or limits complete translocation of the one or more polypeptide through the nanopore. In an embodiment, the positively charged sequence comprises amino acid residues selected from the group consisting of arginine, histidine, and lysine. While these amino acids are described, it will be understood that the positively charged sequence can include additional and/or different amino acid residues.

As above, in an embodiment, the one or more polypeptides comprise an unfoldase slip sequence. In an embodiment, and without being bound by any particular theory, the unfoldase slip sequence is configured to cause the one or more polypeptides to translocate from the first compartment into the second compartment, such as when the unfoldase slip sequence passes through an unfoldase protein, such as a ClpX. As described further herein, the unfoldase slip sequence is believed cause the unfoldase to lose grip of the one or more polypeptides allowing the one or more polypeptides to translocate from the first compartment into the second compartment, i.e., move in a direction opposite that which the unfoldase operates to move the one or more polypeptides. In this regard, the one or more polypeptides, including the unfoldase slip sequence and the analyte sequence, can translocate through the nanopore a plurality of times. Such repeated translocation allows for rereading current patterns from the one or more polypeptides, such as to provide a more accurate sequence call based on the one or more polypeptides.

In an embodiment, the unfoldase slip sequence comprises polyproline sequences. It has been found that such polyproline sequences are configured to cause the unfoldase to lose their grip on polypeptides and allow translocation of the polypeptide through the nanopore.

In an embodiment, the unfoldase slip sequence comprises one or more of a sequence according to SEQ ID NO: 1. In an embodiment, the unfoldase slip sequence comprises one or more of a sequence according to SEQ ID NO: 2.

As discussed further herein, in an embodiment, the one or more polypeptides comprise a plurality of unfoldase slip sequences, such as a plurality of sequences according to SEQ ID NO: 1 and/or SEQ ID NO: 2. In an embodiment, the one or more polypeptides comprise a number of copies of the unfoldase slip sequences, wherein the number is 1, 2, 3, 4, 5, 6, 7, 8, 9, 10, or more. In an embodiment, the one or more polypeptides comprise a number of copies of the unfoldase slip sequences, wherein the number is in a range of 1-10, 1-20, 1-30, 1-40, or more. In an embodiment, the unfoldase slip sequences are directly concatenated, such as where multiple comprise of the unfoldase slip sequence are disposed in a contiguous row in the one or more polypeptide sequence. Without wishing to be bound by any particular theory, it is believed that a greater number of unfoldase slip sequences in a polypeptide increases the chances that the polypeptide will slip in, and consequently translocate through, the unfoldase.

In an embodiment, the unfoldase is a ClpX protein. In an embodiment, the unfoldase comprises an amino acid sequence at least 90% or 95% identical to the amino acid sequence of SEQ ID NO: 3. While ClpX proteins are discussed, it will be understood that other unfoldase proteins will work in this context and fall under the scope of the present disclosure.

In an embodiment, the one or more polypeptides further comprise an ssrA tag. Without wishing to be bound by any particular theory, it is believed that the unfoldase (e.g., ClpX) binds to the ssRA tag, such as a C-terminal tag, and pulls the substrate protein back out of the nanopore. This enables amino acid sequence dependent features of the one or more polypeptides to be analyzed as they are translocated through the nanopore by the unfoldase motor.

In an embodiment, the ssRA tag comprises a sequence according to SEQ ID NO: 5. In an embodiment, the ssRA tag is disposed in a C-terminal direction of the analyte sequence.

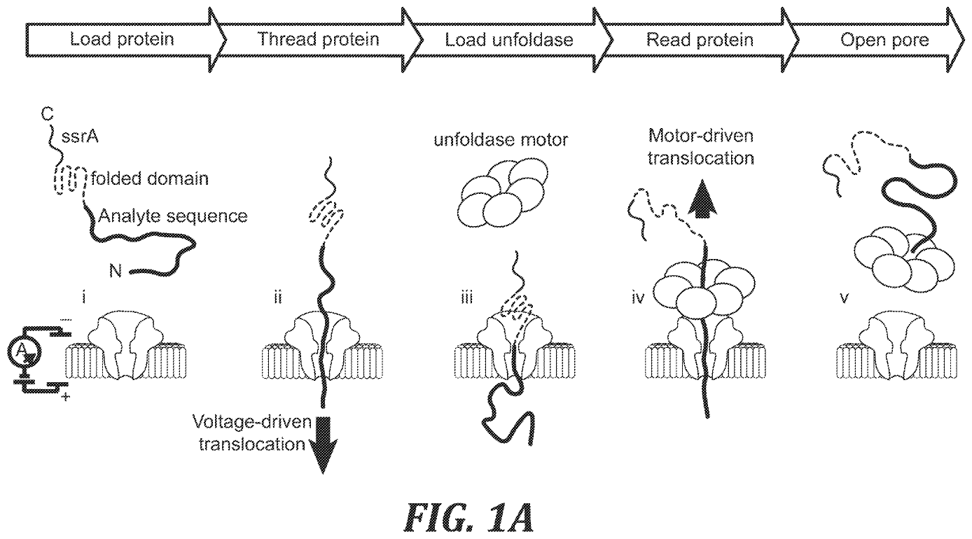

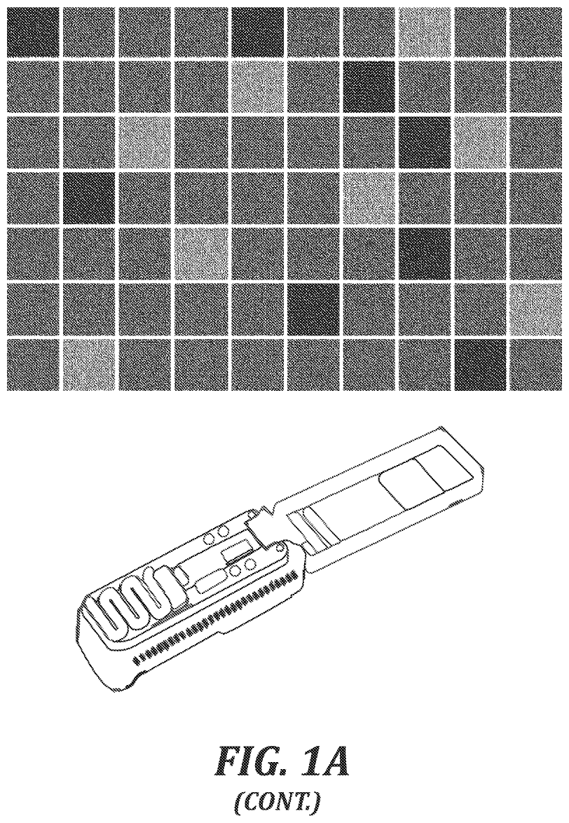

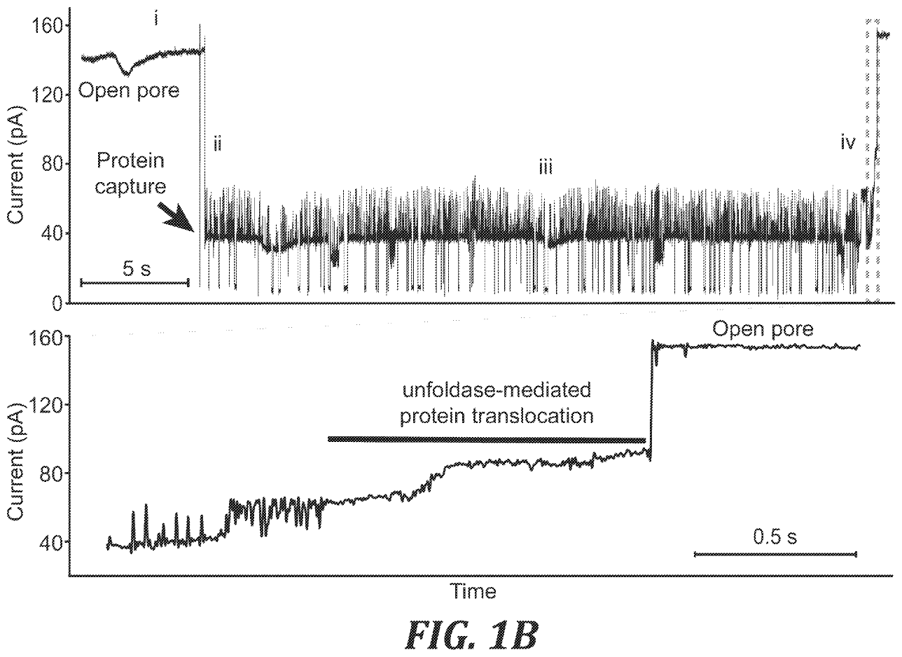

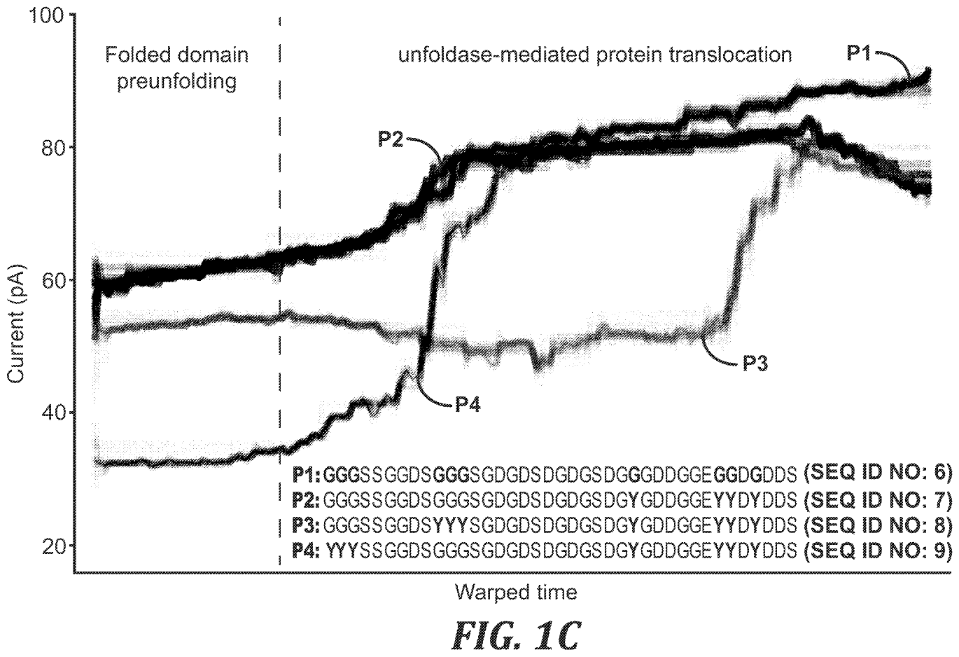

The one or more polypeptides can comprise the various sequences described herein in a number of different configurations and orders. Discussion of such configurations and orders will be discussed relative to a C-terminal and an N-terminal of the polypeptides and the analyte sequence. In an embodiment, the folded domain is disposed in a C-terminal direction relative to the analyte sequence. In an embodiment, the unfoldase slip sequence is disposed in an N-terminal direction relative to the analyte sequence. In an embodiment, the positively charged sequence is disposed in a C-terminal direction relative to the analyte sequence. In an embodiment, the positively charged sequence is disposed in an N-terminal direction relative to the analyte sequence. In an embodiment, the polyanion sequence is disposed in a C-terminal direction relative to the analyte sequence. While such configurations are described and illustrated further herein, such as in FIGS. 1A, 5A, and 7A, it will be understood that other configurations and orders are possible.

As above, in an embodiment, a sample, such as a biological sample, may be analyzed according to the methods of the present disclosure. In an embodiment, the methods of the present disclosure include functionalizing the polypeptides of a sample with the sequences described herein. Accordingly, in an embodiment, the method comprises modifying one or more sample polypeptides to comprise the polyanion sequence; the folded domain, the positively charged sequence; and the unfoldase slip sequence to provide the one or more polypeptides.

In an embodiment, the method includes applying an electrophoretic and/or electroosmotic force between the first compartment and a second compartment of the nanopore sequencing device to translocate partially the one or more polypeptides from the first compartment to the second compartment through a nanopore of the nanopore sequencing device. Such application of electrophorectic force can be applied through the application of a voltage across the first compartment and the second compartment. In an embodiment, the voltage is a constant voltage. In an embodiment, the voltage is a voltage in a range of about ±100 mV to about ±200 mV. In an embodiment, the voltage is a voltage in a range of about ±110 mV to about ±190 mV. In an embodiment, the voltage is a voltage in a range of about ±120 mV to about ±180 mV. In an embodiment, the voltage is a voltage in a range of about ±130 mV to about ±170 mV. In an embodiment, the voltage is about ±140 mV. In an embodiment, the voltage is about ±180 mV.

In an embodiment, the method comprises translocating, with an unfoldase enzyme, the one or more polypeptides partially through the nanopore from the second compartment to the first compartment. In this regard, the unfoldase opposes the electrophoretic or electroosmotic forces to translocate the one or more polypeptides back into the first compartment.

In an embodiment, the method comprises measuring a current corresponding, such as temporally corresponding, to moving, with the unfoldase enzyme, the one or more polypeptides from the second compartment to the first compartment.

The methods of the present disclosure can include analysis of current patterns generated as the one or more polypeptides, including the analyte sequence, pass through the nanopore, such as when the one or more polypeptides pass repeatedly through the nanopore. As above, the methods of the present disclosure comprise measuring a current corresponding to moving, with the unfoldase enzyme, the one or more polypeptides from the second compartment to the first compartment. In an embodiment, a current pattern from the measured current corresponds to translocating, with the unfoldase, the one or more polypeptides at least partially from the second compartment to the first compartment a plurality of times. In an embodiment, the current pattern can indicate or is otherwise based upon a primary sequence of the one or more polypeptides passing through the nanopore.

As discussed further herein, in certain instances, the one or more polypeptides translocate through the nanopore a plurality of times, such as due to the unfoldase losing grip on the unfoldase slip sequence. Accordingly, in an embodiment, the methods of the present disclosure can comprise combining portions of the current pattern corresponding to translocating the one or more polypeptides, particularly portions corresponding to the analyte sequence, from the second compartment to the first compartment to provide a combined current pattern.

In an embodiment, the methods comprise comparing a portion of the combined current pattern corresponding to the analyte sequence to a reference current pattern obtained using a reference polypeptide measured under analogous conditions. Such analogous conditions can include, for example and without limitation, temperature, compartment pH, compartment salinity, unfoldase concentration, number of unfoldase slip sequences per polypeptide, applied voltage, electrophoretic force, electroosmotic force, and the like. By comparing the combined current pattern with reference current patterns, a sequence call can be generated to determine or estimate a primary sequence of the one or more peptides, such as including the analyte sequence. Accordingly, in an embodiment, the method comprises generating an analyte sequence call based on comparing the portion of the combined current pattern corresponding to the analyte sequence to reference current pattern the reference polypeptide analyte measured under analogous conditions.

In another aspect, the present disclosure provides kit for analyzing a polypeptide, such as for analyzing a sequence of a polypeptide. In an embodiment, the kit comprises a nanopore sequencing device comprising a first compartment; a second compartment; a nanopore disposed in an insulating membrane fluidically separating the first compartment from the second compartment; an unfoldase disposed in either the first compartment or the second compartment; and one or more unfoldase slip sequences.

As discussed further herein, in an embodiment, the unfoldase can lose grip of the unfoldase slip sequence, and, consequently, any polypeptide coupled to the unfoldase slip sequence. In this way, the kits of the present disclosure are configured to translocate repeatedly the unfoldase slip sequence and polypeptides coupled thereto to provide repeated reads of the polypeptides and provide a more accurate read of such polypeptides.

In an embodiment, wherein the unfoldase slip sequence comprises polyproline sequences. In an embodiment, the unfoldase slip sequence comprises one or more of a sequence according to SEQ ID NO: 1. In an embodiment, the unfoldase slip sequence comprises one or more of a sequence according to SEQ ID NO: 2.

In an embodiment, the kit comprises reagents for coupling or otherwise modifying one or more sample polypeptides to comprise a polyanion sequence; a folded domain, a positively charged sequence; and a unfoldase slip sequence, as discussed further herein to provide one or more polypeptides suitable for analysis in the kits of the present disclosure. In an embodiment, the kit comprises reagents configured to functionalize a sample polypeptide with the one or more unfoldase slip sequences.

In an embodiment, the nanopore sequencing device comprises a voltage source configured to apply a voltage across the first compartment and the second compartment, such as to translocate a polypeptide through or partially through the nanopore.

In an embodiment, the nanopore sequencing device comprises a current sensor configured to measure current passing through the nanopore, such as when the voltage source is applying a voltage, such as to generate a signal based the current and to generate a sequence call based on the current.

### EXAMPLES

### Example 1: Methods

### Expression and Purification of Proteins

Plasmids for analyte proteins were constructed with gBlocks (Integrated DNA Technologies) inserted into the pET-49b(+) plasmid (Novagen), with a dihydrofolate reductase domain, a polyhistidine tag, and a TEV cleavage site upstream of the sequence encoding an analyte protein. The NEBuilder HiFi DNA Assembly and Q5 site-directed mutagenesis kits (New England Biolabs) were used for plasmid construction. Cloning was performed using NEB 5-alpha competent E. coli cells. Plasmid sequences were verified by Sanger sequencing through Genewiz. Protein expression was induced overnight at 30 °C with BL21 (DE3) E. coli cells in Overnight Express Instant TB medium (Novagen). Proteins were purified via immobilized metal affinity chromatography (IMAC) with TALON metal affinity cobalt resin and its associated buffer set (Takara), following the manufacturer's instructions. Proteins were cleaved with TEV protease (New England Biolabs) and further purified via reverse IMAC. Purified proteins were concentrated using ultracentrifugal filters with a 10 kDa cutoff (Amicon) and stored over the short term at 4 °C or over the long term at −80 °C until use.

A covalently linked hexamer of an N-terminal truncated ClpX variant (ClpX-ΔN₆) was prepared using the BLR E. coli strain as described previously. In brief, cells were grown to an A₆₀₀ of ~0.6 in LB medium and then incubated in the presence of 0.5 mM isopropyl β-D-1-thiogalactopyranoside (IPTG) at 23 °C for ~3 h to induce ClpX expression. ClpX was purified via IMAC and anion exchange chromatography. Purified ClpX was stored at −80 °C in small aliquots until use. ClpP expression was induced at an A₆₀₀ of ~0.6 with 0.5 mM IPTG at 30 °C for ~3 h⁴⁰. ClpP was purified by IMAC and stored at −80 °C until use.

### PTM Assays

For asparagine deamidation, protein (~1 mg/mL) was incubated overnight in 100 mM sodium bicarbonate buffer (pH 9.6) at 25 °C to catalyze deamidation. For protein phosphorylation with kinase, protein was incubated either with 50,000 units/mL of PKA (New England Biolabs) or 10,000 units/mL of CKII (New England Biolabs) in a protein kinase buffer (10 mM MgCl₂, 0.1 mM EDTA, 2 mM DTT, 0.01% Brij 35, 260 μM ATP and 50 mM Tris-HCl, pH 7.5) at 30 °C. The protein solution was used for nanopore analysis immediately after the incubation without purification.

### MinION® Experiments

All experiments were performed on the MinION® platform using R9.4.1 flow cells. Run conditions were set with a custom MinKNOW® script (available from Oxford Nanopore Technologies) at a temperature of 30 °C and a constant voltage of −140 mV with a 3 kHz sampling frequency, except for initial proteins P1-P4, in which runs were performed at a constant voltage of −180 mV with a 10 kHz sampling frequency. Using the priming port, flow cells were first washed with 1 mL running buffer (200 mM KCl, 5 mM MgCl₂, 10% glycerol, and 25 mM HEPES-KOH, pH 7.6) and then loaded with 200 μL of protein analyte in running buffer at a final concentration of 500 nM unless otherwise specified. Following observation of protein captures in the pores, flow cells were washed with 1 mL of running buffer to remove uncaptured proteins and subsequently loaded with 75 μL of running buffer supplemented with 4 mM ATP and 200 nM of ClpX-ΔN₆ unless otherwise specified. The flow cell was washed ~4 min after the analyte loading in the initial method while the flow cell was washed ~6 min and ~2 min after the loading of the analyte at the concentration of 5 nM and 500 nM, respectively, in the optimized method. For MinION® runs in the high salt condition, a buffer containing 400 mM KCl, 5 mM MgCl₂, and 25 mM HEPES-KOH (pH 7.6) was used in place of standard running buffer.

### Bulk Degradation Assays

The time-course degradation assay of the PASTOR-HDKER protein was performed in running buffer with 6 μM PASTOR-HDKER, 150 nM ClpX-ΔN₆, 300 nM ClpP₁₄, and an ATP regeneration mix (4 mM ATP, 16 mM creatine phosphate, and 7 units/mL creatine phosphokinase) at 30 °C. Incubation was stopped by denaturing samples in the Laemmli buffer at 95 °C for 5 min. Samples were run on SDS-PAGE and stained with Coomassie blue to quantify protein bands using the ImageJ software.

### Nanopore Signal Analysis

Preprocessing: To assist in identifying ClpX-mediated protein translocations, detection thresholds were established using specific statistical parameters (standard deviation, median value, standard deviation of the mean of windows, and the ratio of values relative to the open pore value) indicative of translocation to ionic current blockades preceding a return to open channel. This analysis was used to assist the process of manually checking traces for translocations, and translocations with substantially high noise or disruptions were discarded. PASTOR proteins were auto-segmented as described below, with the exception of PASTOR proteins containing folded domains and PASTOR-reread, which were manually segmented. PASTOR-reread rereads with a complete Y₂-Y₃-Y₄-Y₅-Y₂ signal were assumed to be full-length reads with a back-slipping distance of 310 aa. Partial rereads missing the signal(s) of the C-terminal Y₂, Y₃, Y₄, and Y₅ were assigned to back-slipping distances of 250, 188, 125, and 61, respectively. All figures with raw traces (those shown in pA) had a low-pass Bessel filter applied using SciPy with N=10 and Wₙ=0.025. Before use in data analysis, transformed traces (as in FIG. 1C, 2C-2E, 2G, 4, 5F-5G, 7D, 7F) by applying a low-pass Bessel filter with N=10 and Wₙ=0.03 with scipy, average downsampling by a factor of 50 for proteins P1-4, 20 for the 8 PASTORs, and 10 for the other proteins, and then scaling. To scale, the segment was split into tenths, and the median of the minimums of each tenth and the median of the maximums of each tenth were used as the min and max, respectively, to perform min-max scaling. For PASTOR-phos, the signals were iteratively scaled. This approach was first used then DTW aligned traces to two canonical pre-segmented traces and selected the alignment with the lowest DTW distance. The max value of the N-terminal VR was multiplied by 1.4 and the max value of VR GLSARRL was multiplied by 1.2, and the minimal max was used as the max value for min-max scaling. This was repeated a second time after re-aligning to the canonical traces and segmenting the VRs. Unless otherwise specified, ‘normalized’ refers to z-score normalization, as in ‘normalized current’ when comparing a model signal to experimental signals.

Signal alignment: To align signals, we used DTW, and normalized DTW distances by dividing by the sum of the lengths of the two signals. To describe the similarity of a set of traces, the DTW distance was computed between all pairs of traces. In t-SNE plots, we then clustered traces on the vector of its DTW distances to all other traces. To create ensemble traces, we first identified the trace with the lowest mean DTW-distance to all other traces and stretched it to create T_{medoid}=[t₁, t₂, . . . , tₙ], where n is the mean length of all traces. We then DTW-aligned every other trace to T_{medoid}, and created T_{consensus}=[median(alignments to t₁), median(alignments to t₂), . . . median(alignments to tₙ)]. Ensemble traces in FIGS. 1C, 6B, and 7D show all traces aligned to the T_{consensus}, but do not plot T_{consensus}.

Protein sequence-to-signal model: To describe the amino acids, their volumes and their charge at pH=7.6 were used, where the histidine residue is assumed to be neutral. Phosphoserine's volume was estimated as 126.6 cm³/mol, based on a linear regression of MW to volume of the other residues. The model signal of a sequence [aa₁, aa₂, . . . , aaₙ] is calculated by computing the signal, Si, for each of the n-19 windows of width 20. The vector Xᵢ describes the window starting at index i in the sequence. The j^{th} index in Xᵢ is 1+V_{c}*Volume(aaᵢ₊ⱼ)+P_{c}*PositiveCharge(aaᵢ₊ⱼ)+N_{c}*NegativeCharge(aaᵢ₊ⱼ), for 0≤j<20, where the functions PositiveCharge and NegativeCharge take 1 if the residue has a positive or negative charge, respectively, and 0 otherwise. To weight the values in Xᵢ, we use a vector PW of length 20 containing values representing a negative, centrally positioned parabolic curve. Sᵢ is then computed as the dot product of Xᵢ and PW. The weights between charge and volume, V_{c}=−3.9×10⁻³, N_{c}=4.08×10⁻¹, and P_{c}=−8.16×10⁻², were determined empirically to minimize the DTWDS of a training subset of protein traces.

ClpX Step Identification: For this analysis, the signals were not scaled nor downsampled. They were filtered with a low-pass bessel filter with N=10 and Wₙ=0.7. For this analysis, YY dips were manually extracted, including portions of the signal that would otherwise be considered part of the VR in this study, to best capture the entire portion where the double tyrosines contribute to the signal. The number of residues per YY dip was calculated as

$$\frac{pw}{d} ,$$

where p=mean proportion of the total translocation dwell time spent in these regions (0.318), w=total number of reading windows in the sequence (359), and d=number of YY dips per read (6). A total of 776 YY dip regions were analyzed, which is 45% of all YY dips in the dataset, omitting dips affected by potential backstepping (non-monotonic steps) or excessive noise. When using the Bayesian-based algorithm, a minimum length of 10 observations and a threshold of 18 was used. When using the t-test based algorithm, a minimum window length of 10 observations and a threshold p-value of 5×10⁻⁵ were used.

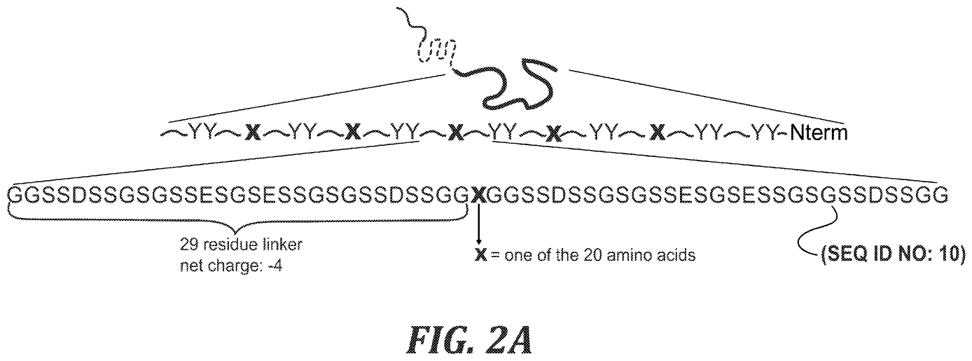

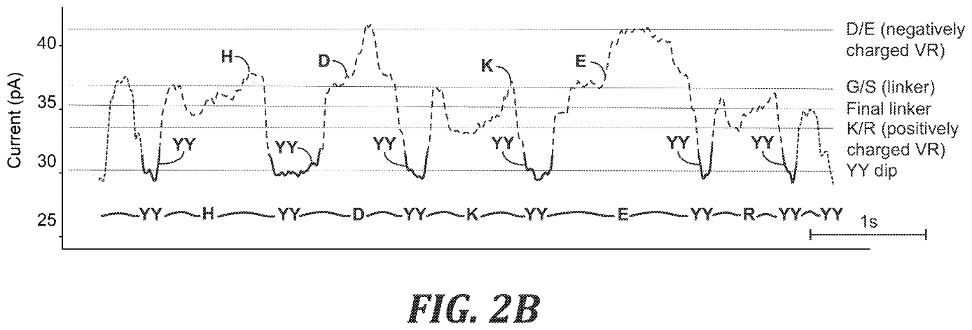

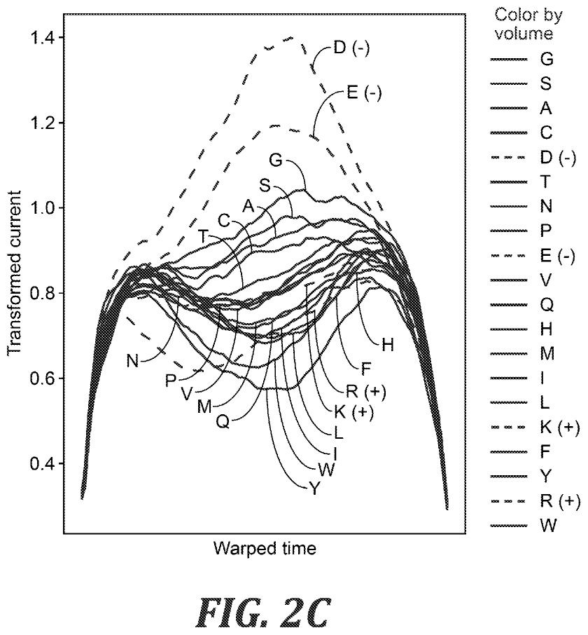

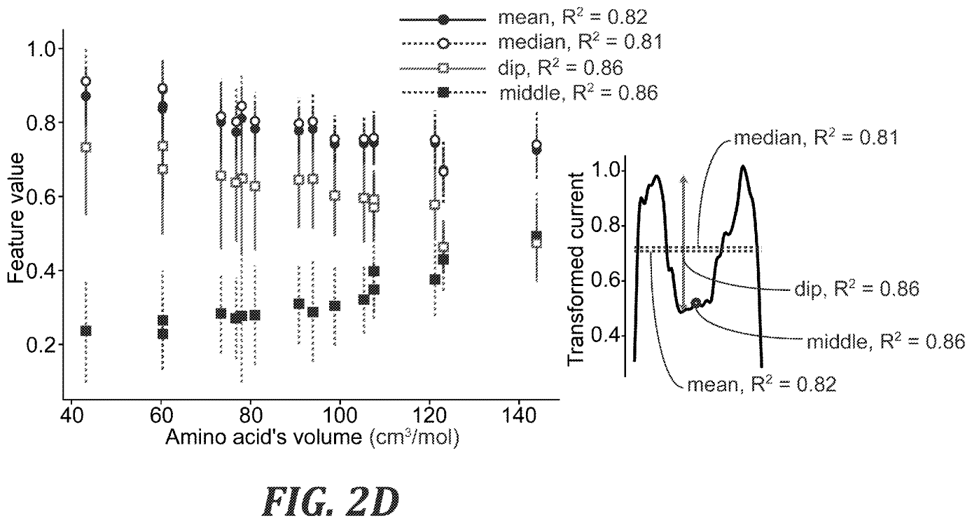

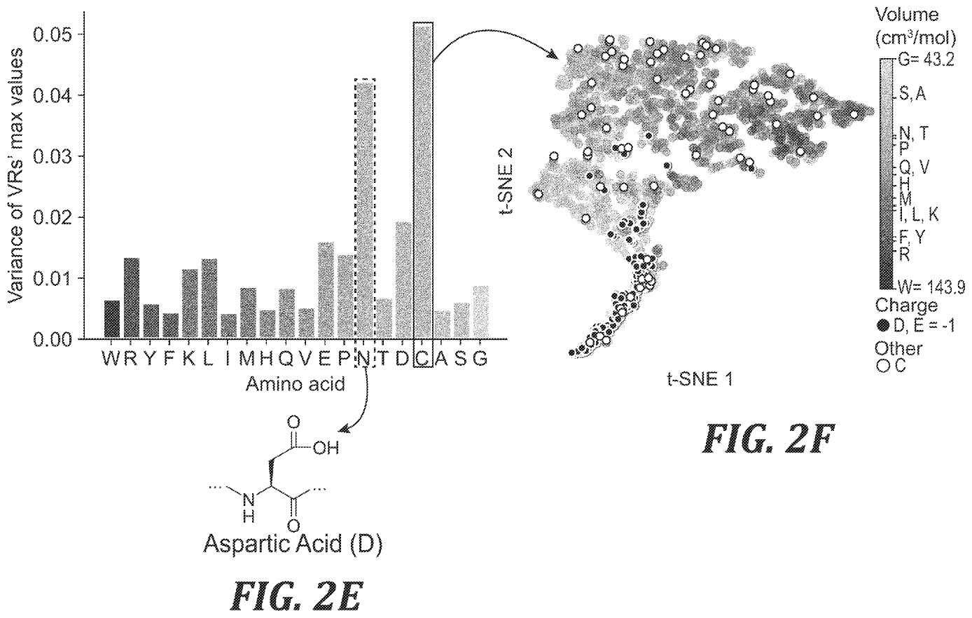

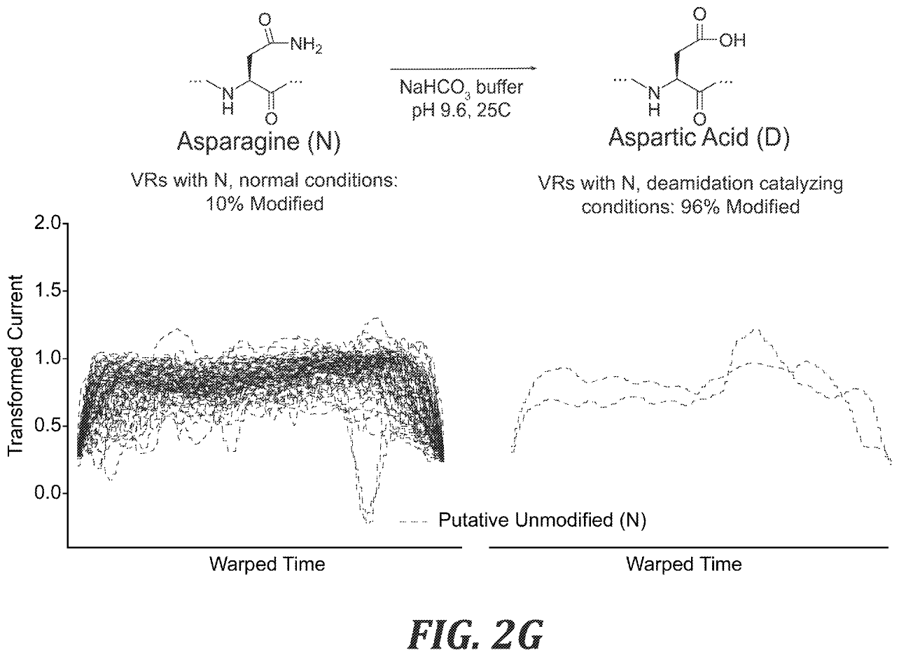

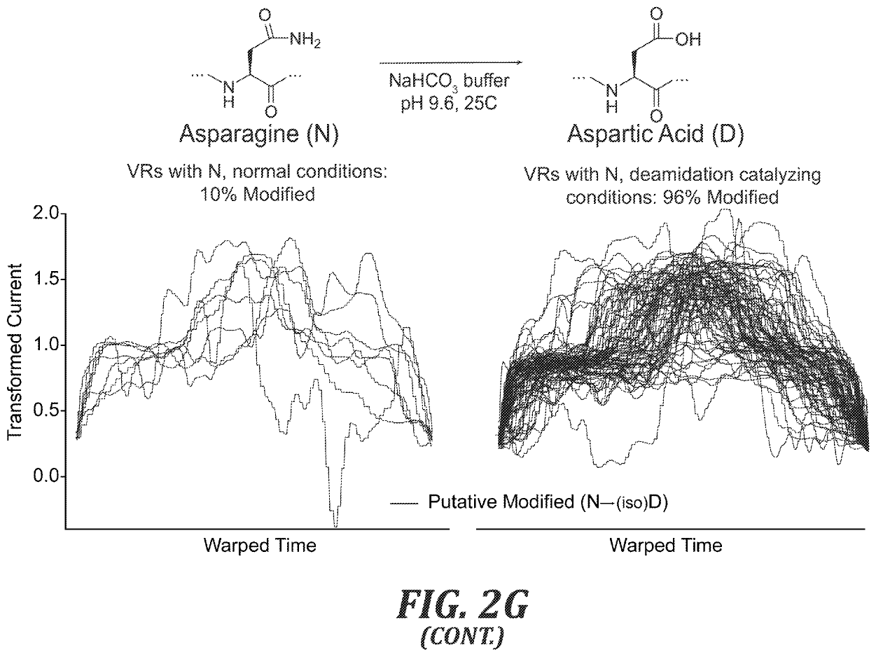

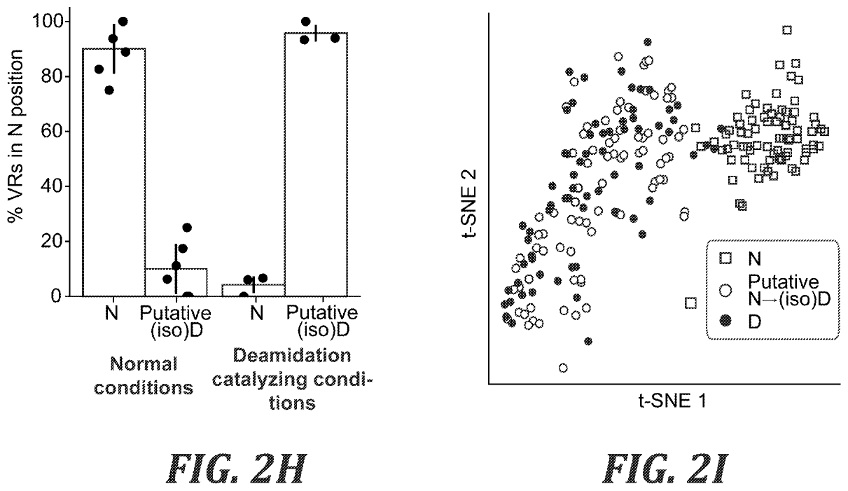

YY-Segmentation: To identify the YY dips and VRs, a single PASTOR trace was manually segmented into each section shown in FIG. 2A, and the remainder of the traces were aligned to it with DTW. The corresponding regions were assigned the label from the one manually segmented trace. For PASTOR-phos, two canonical traces were manually segmented, and the rest of the traces were aligned to both, and then labels were assigned based on the canonical trace with the lowest DTW distance.

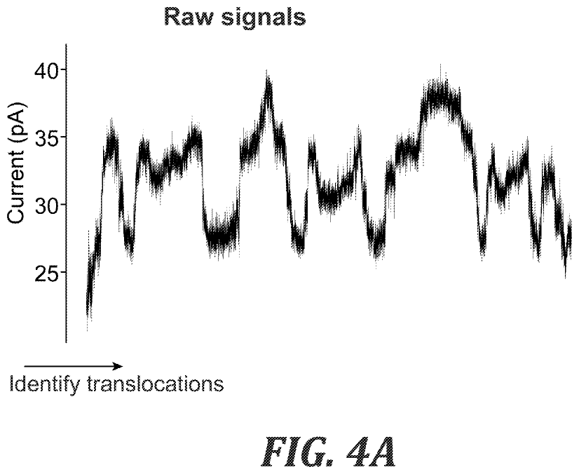

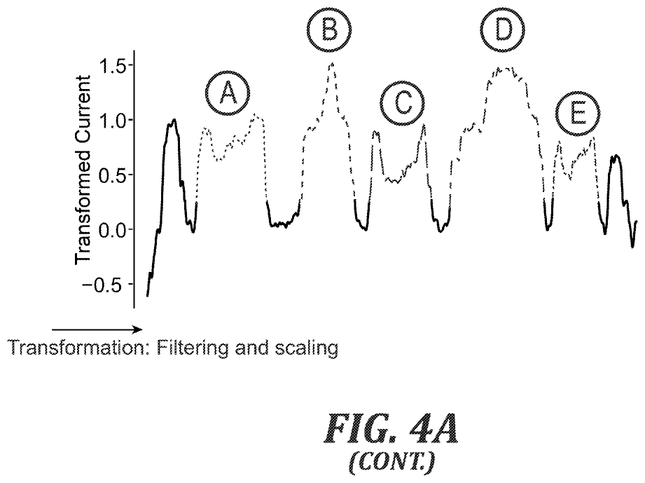

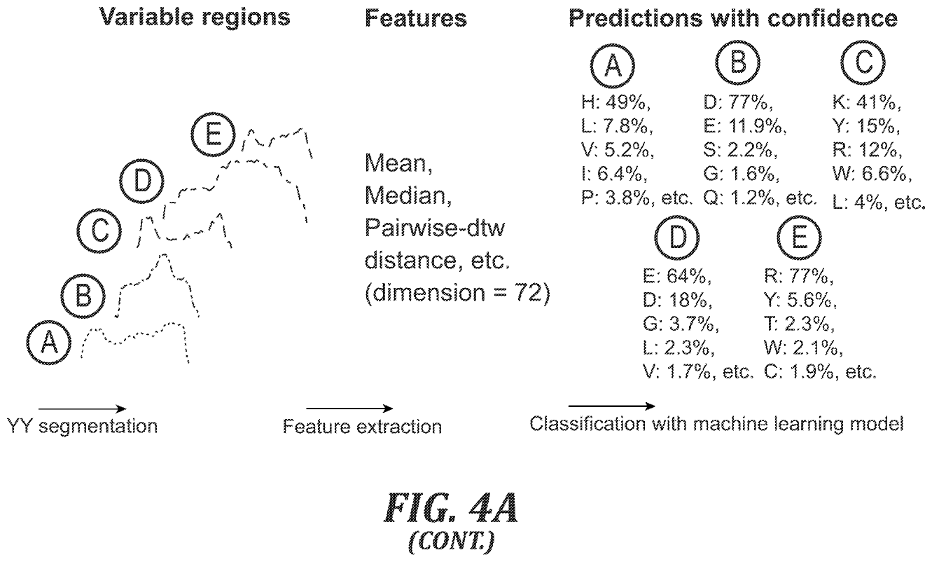

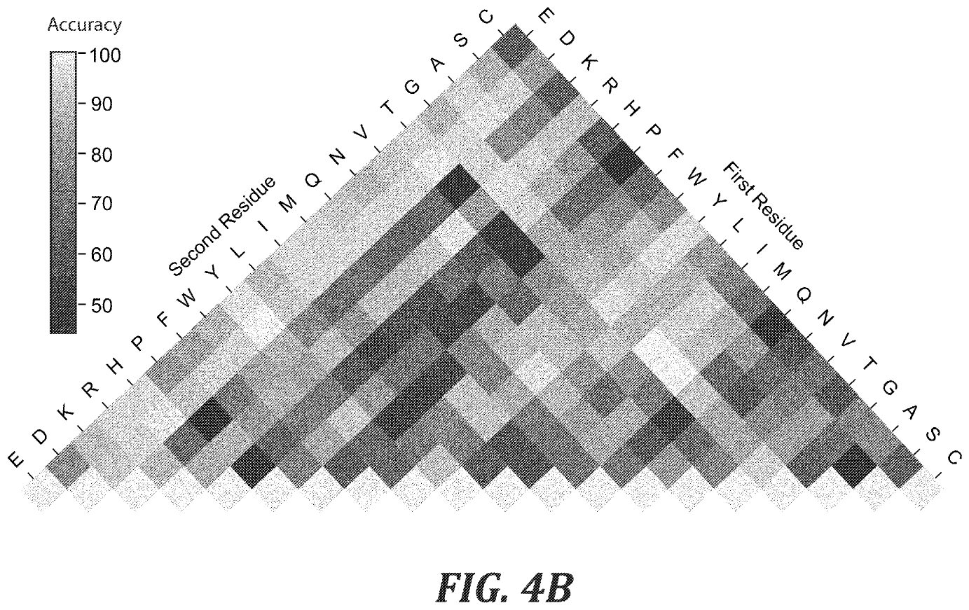

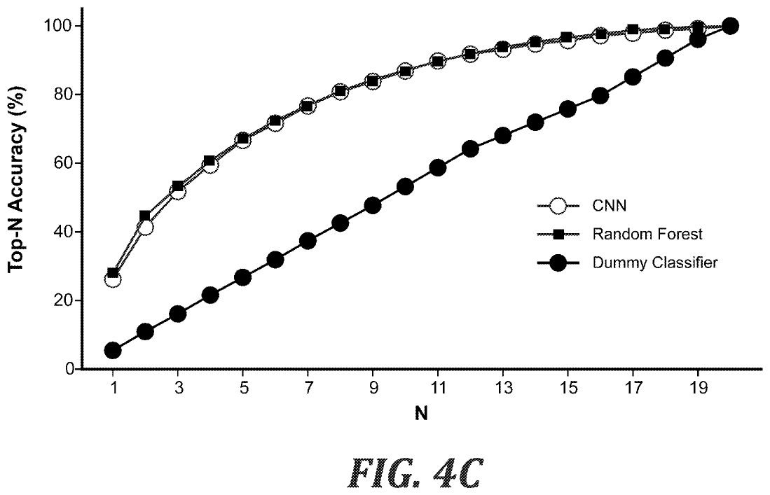

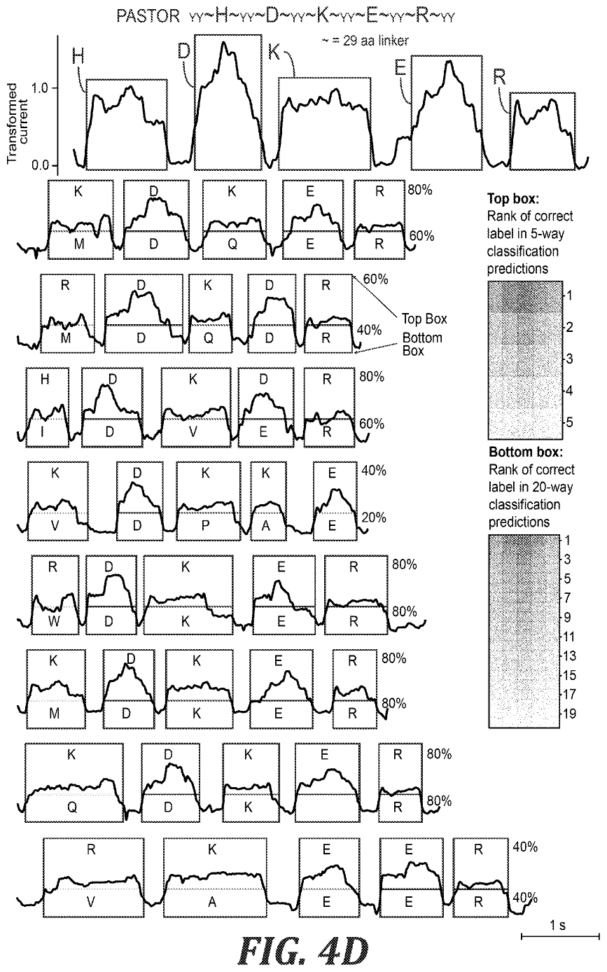

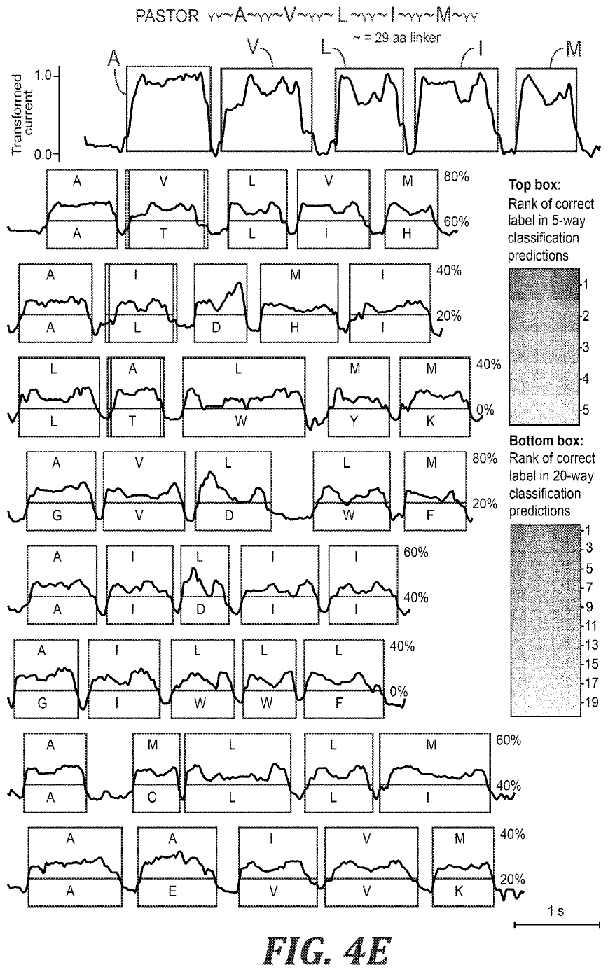

VR classification: scikit-learn was used to develop and test classical machine learning models and Pytorch to develop and test convolutional neural network models. The test set was composed of all current traces from a given set of experiments to create an out-of-sample test set. The set of test experiments was selected using linear programming (Python package Pulp) to ensure at least 12 VRs with each amino acid in the test set, while minimizing the test set size. The number 12 was selected because it gave the closest to an 80-20 train-test split. 79.6% of the VRs were in the training set, and 20.4% were in the testing set. In classification tasks where only VRs corresponding to a subset of amino acids were used, the test set is composed of a subset of this test set. Hyperparameter tuning was performed with scikit-optimize on the training set using 5-fold cross validation. The optimal parameters were n_estimators=250, min_samples_leaf=2, max_features=‘log2’, max_depth=20, ccp_alpha=0.0001, class_weight=‘balanced_subsample’, and criterion=‘gini’. All results from FIGS. 4B-4E are from models evaluated on the test set. All VRs containing an asparagine with a maximum transformed value above 1.3 had their labels changed to aspartate. In training all classical models, minority classes were upsampled, such that there was an equal representation of all classes in the training set. When training the CNN in FIG. 2C, the loss inverse proportion of the label's class's representation was weighed in the training set. To featurize the VRs, PCA was performed on the vector of its DTW-distances to all VRs in the training set, to reduce the size of the vector to 64. The median, max, middle, mean, dip, mean absolute value of the derivative, and median absolute value of the derivative of the transformed signals was used, in addition to the standard deviation of the raw (unfiltered, unscaled) signal. The CNN had the transformed signal as input. It was trained with a stochastic gradient descent optimizer with a learning rate of 0.01, had four convolutional layers followed by a GRU and then a fully connected layer, and was initialized with Kaiming initialization. Max pooling and a ReLU activation function were applied after each convolutional layer. The dummy classifier was implemented with scikit-learn's dummy classifier with default parameters.

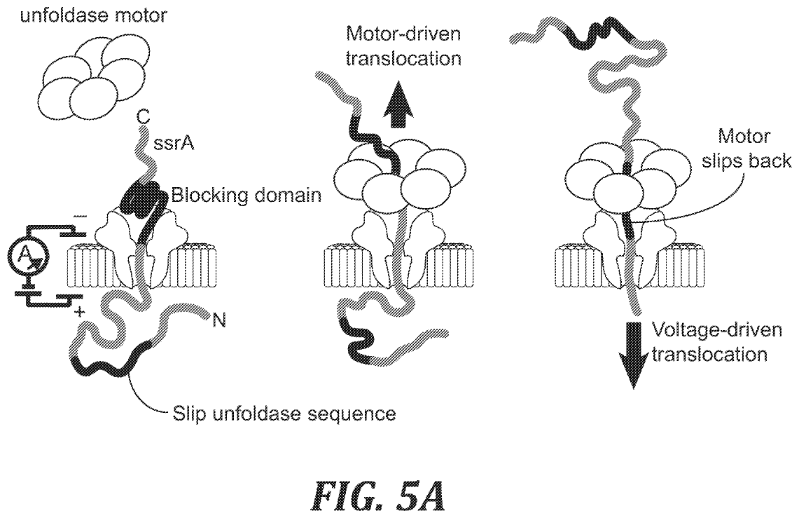

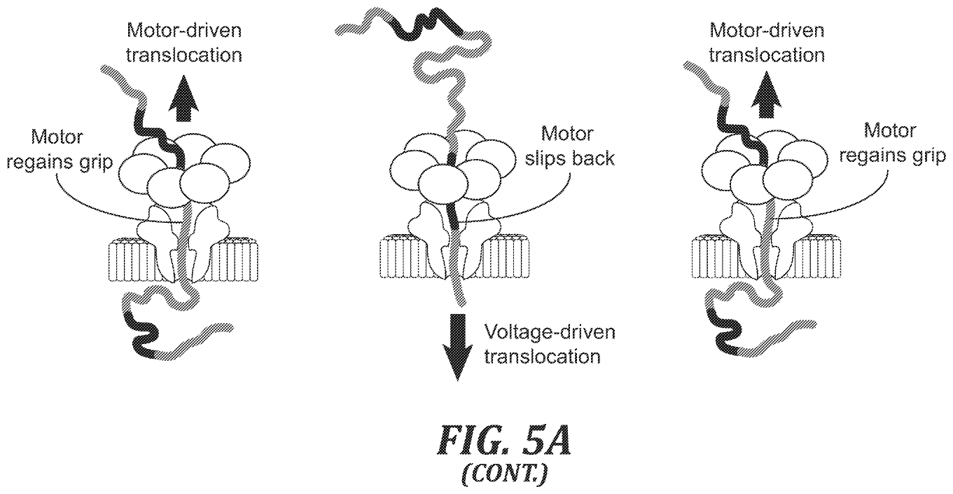

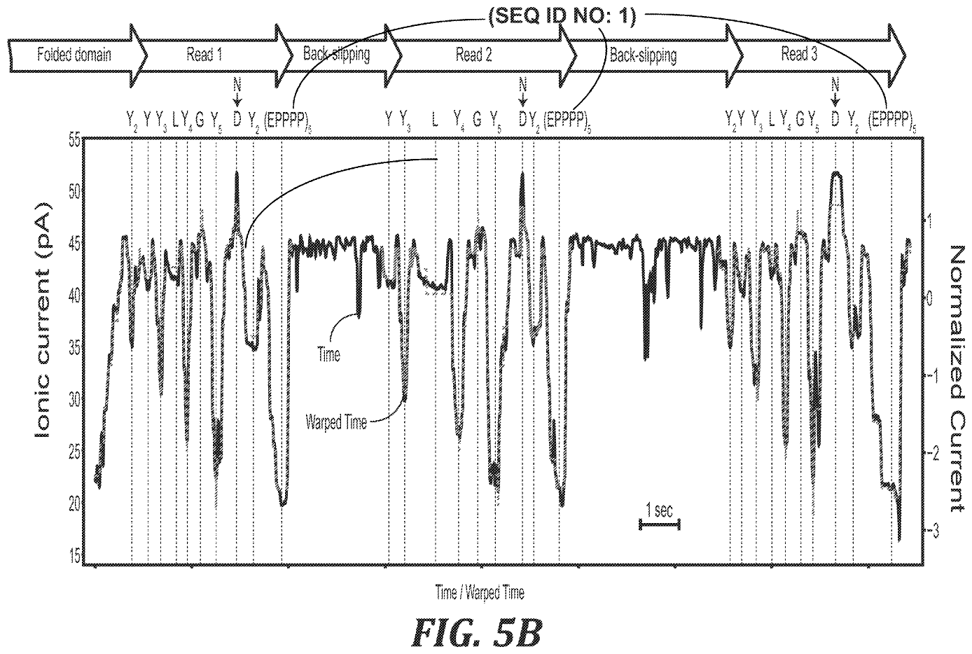

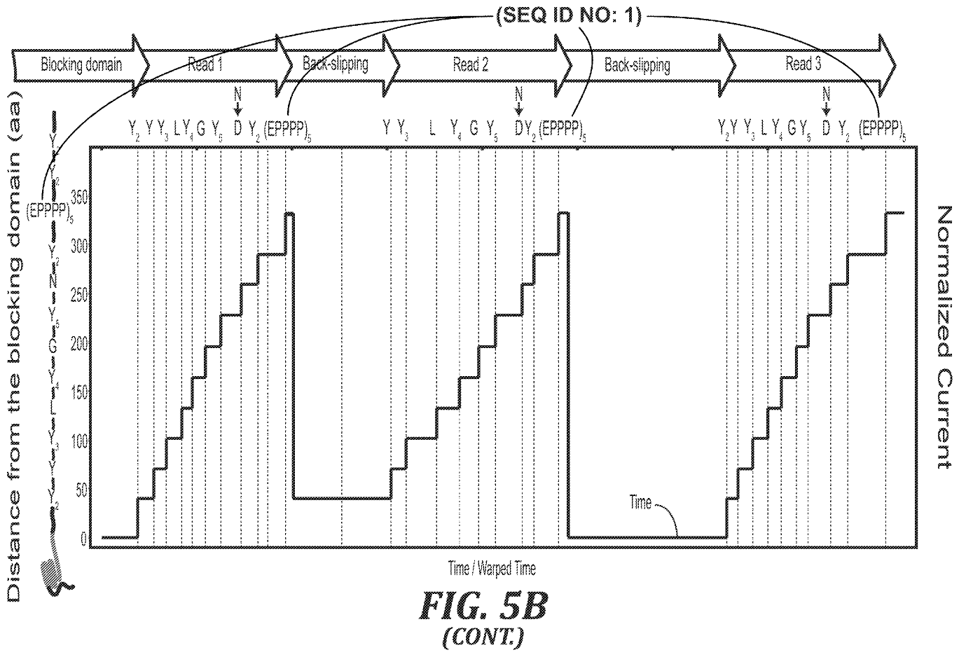

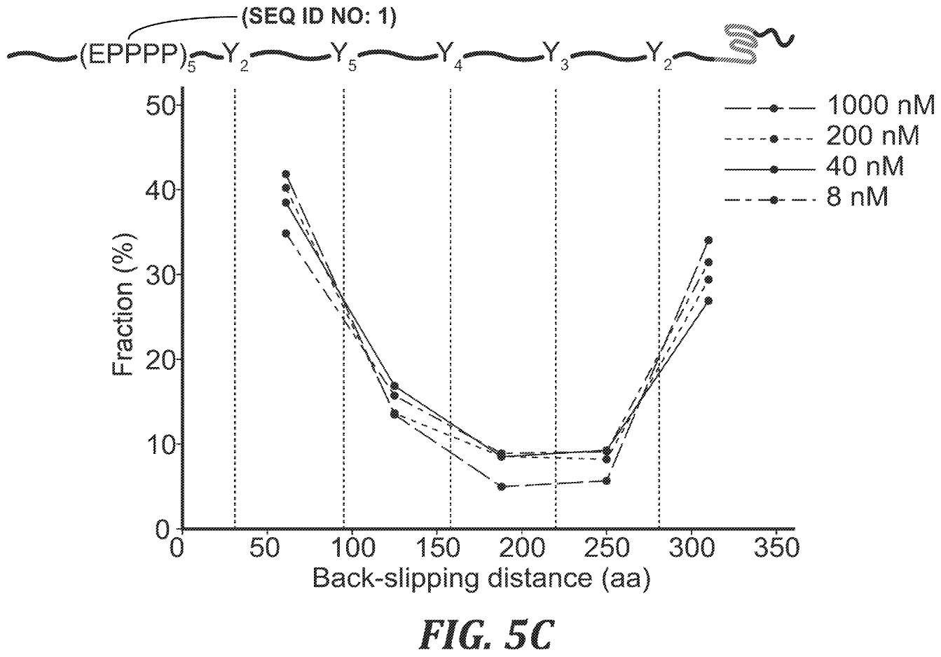

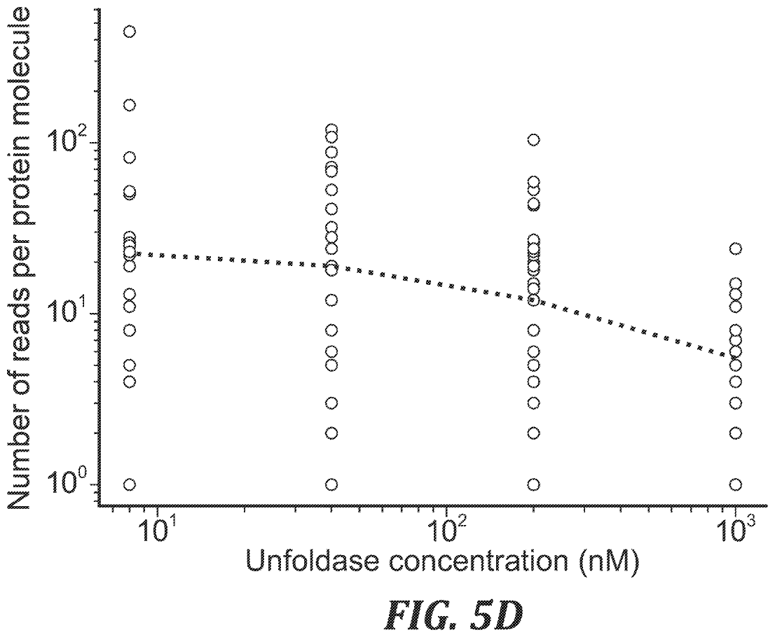

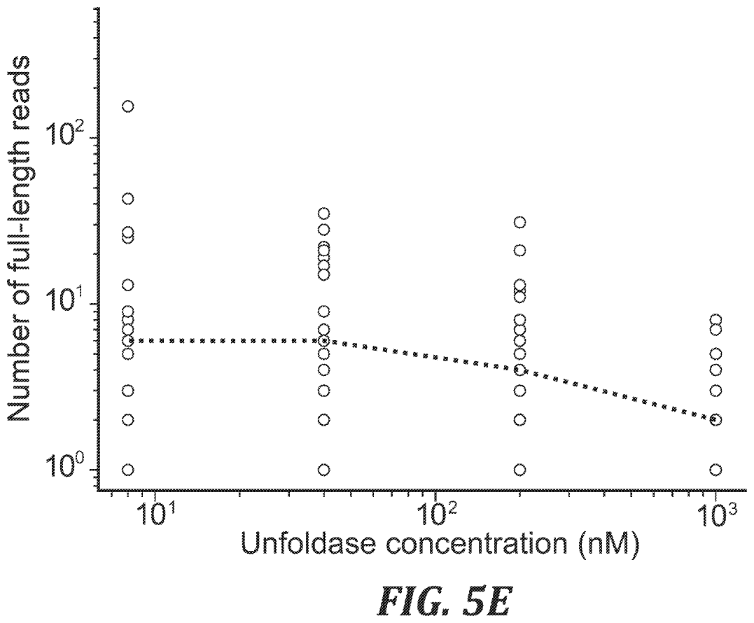

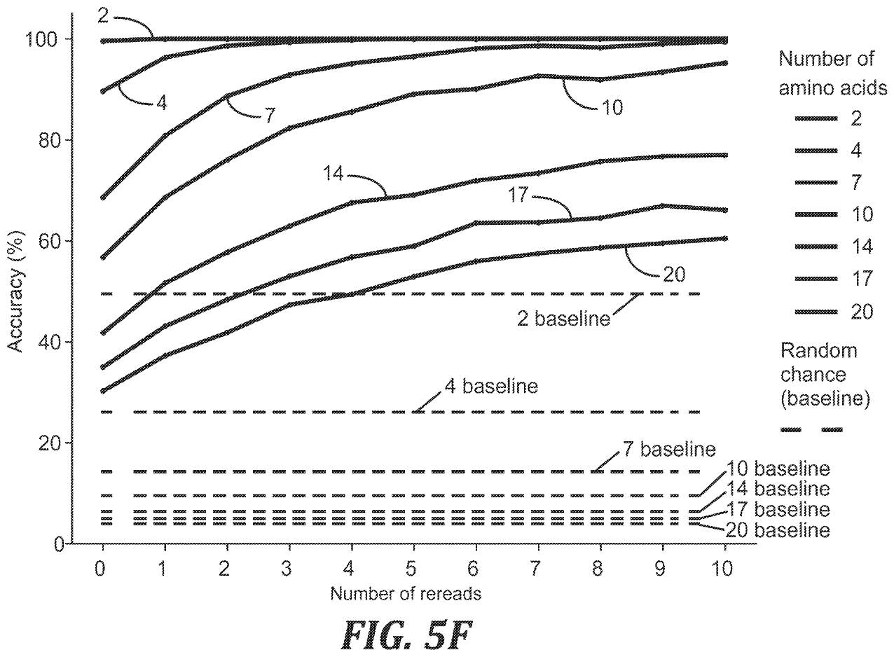

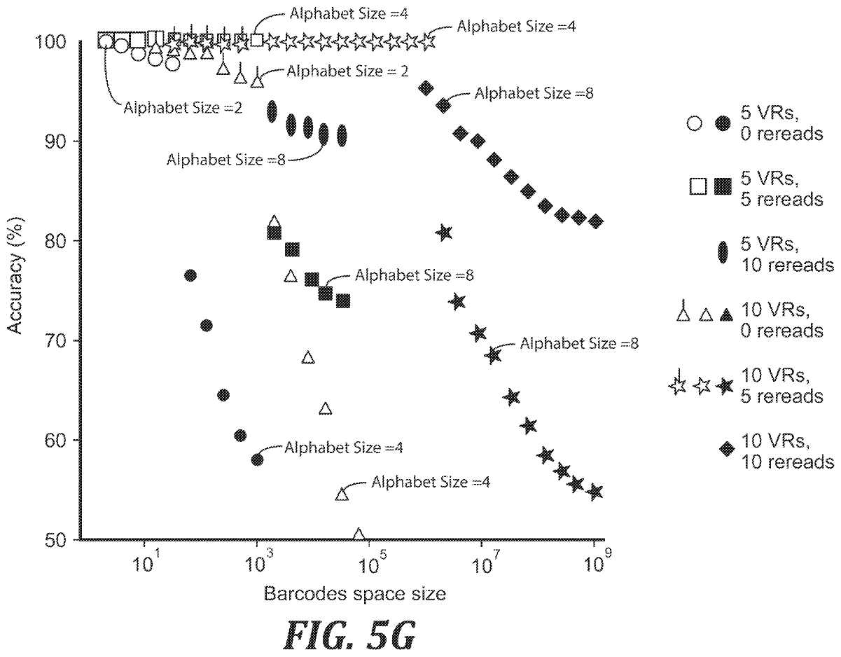

Reread simulation: The results from FIGS. 5F and 5G are using random forest without hyperparameter tuning, and are the results from 100 randomly selected 80-20 train-train splits. This was used to estimate the accuracy with a large number of rereads, given the data limitation and the need to group samples in the test set.

Barcode error correction: To calculate the accuracy of barcode identification when using linear error correcting codes, we started with our accuracy, p_{VR}, of identifying a VR given an alphabet size, a, of 2, 4, 8, and 16. For a given a and number of VRs, L, we calculated the number of bits n=L×log₂(a) that could be encoded in a protein. We simulated the accuracy with error correction, p′, when n-k of the bits were allocated to linear error correcting codes, for all integers k=1 to n. This was done by conducting 50,000 trials of 1) encoding a random integer from 0 to 2k with a generating matrix into a message of n bits; 2) randomly, independently, with probability p_{VR}, changing each of the

$$\frac{n}{{log}_{2} ( a )}$$

consecutive sets of log₂(a) bits in the encoded message (to a different set of bits of the same length) to simulate misclassifying one VR; and 3) decoding the number with syndrome decoding. p′ was calculated to be the percent of trials where the decoded number equaled the original random number.

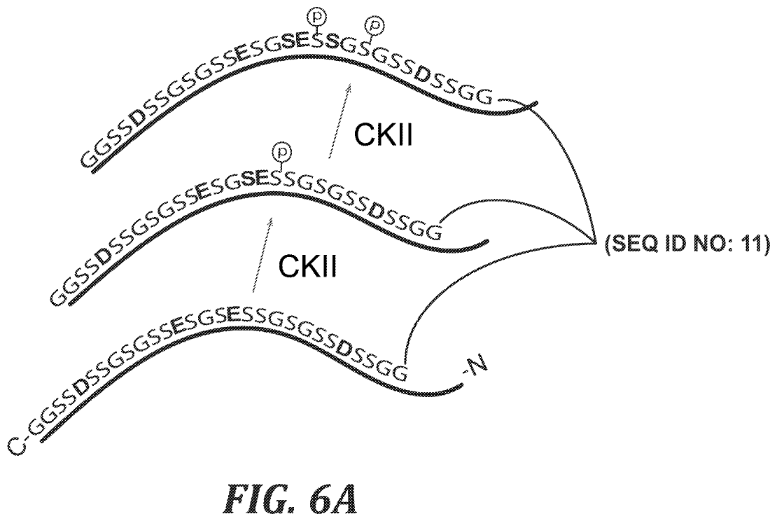

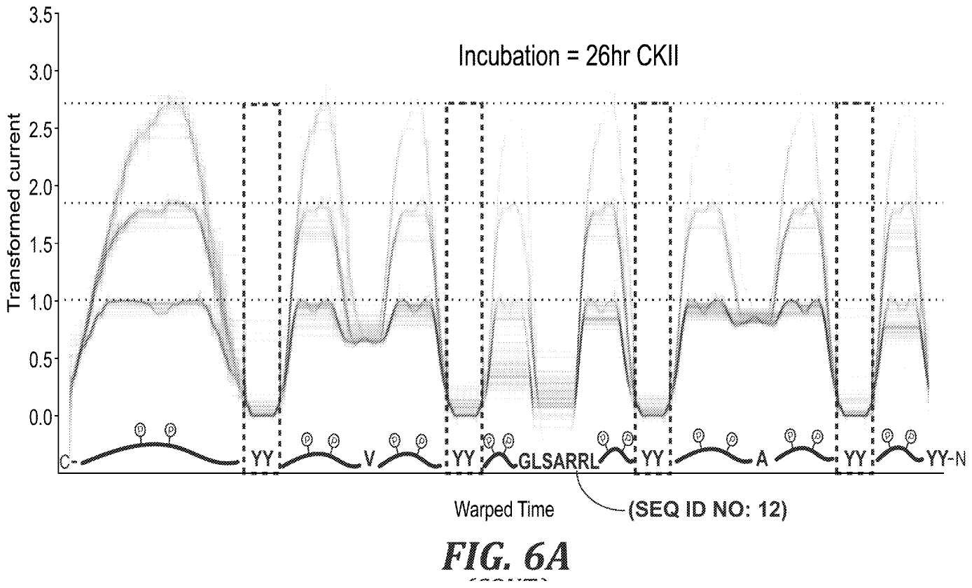

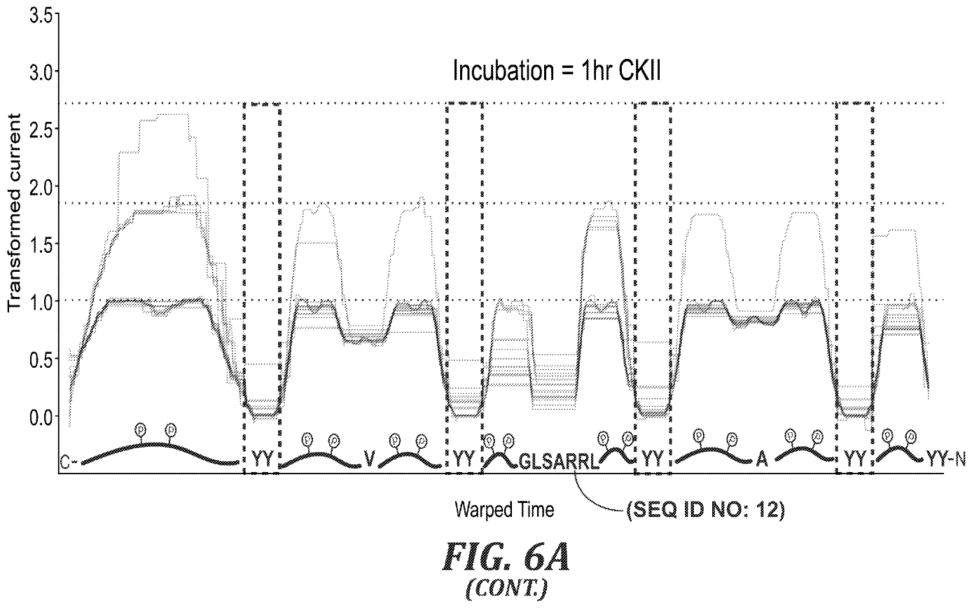

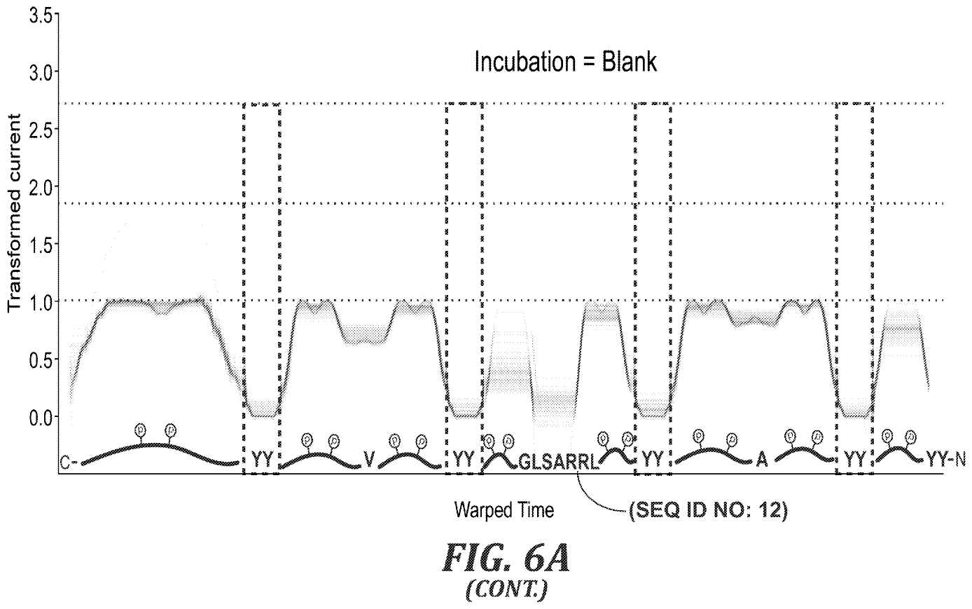

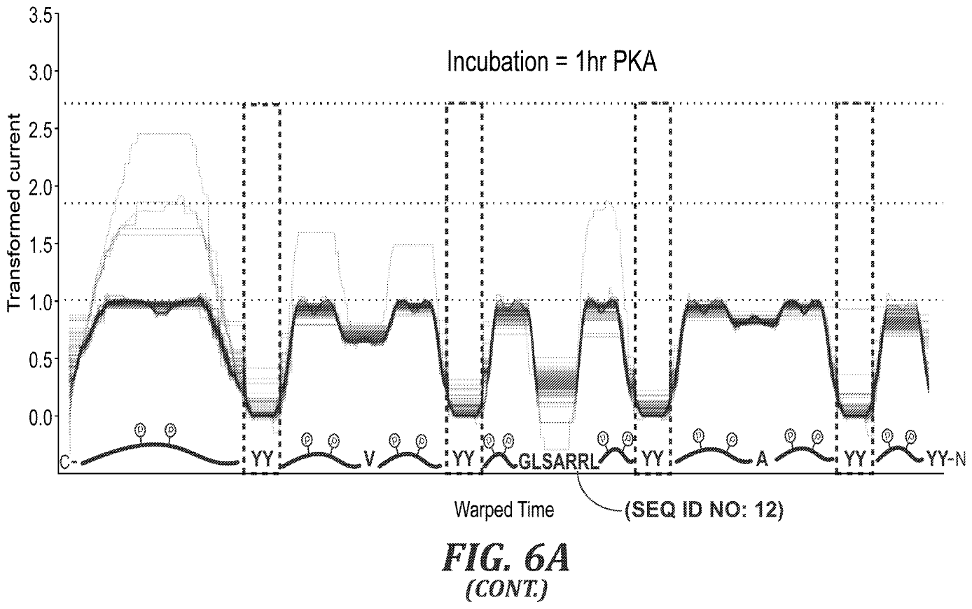

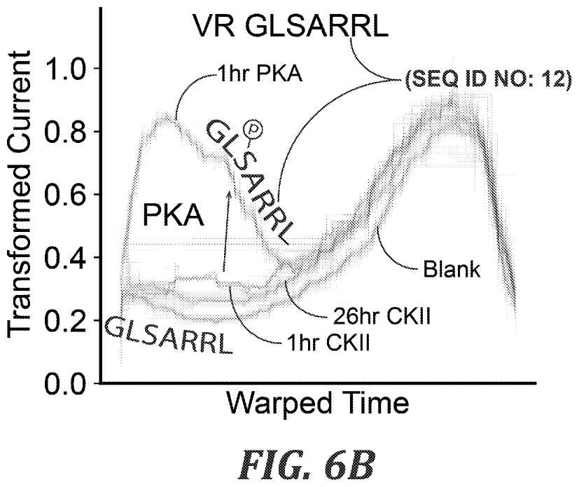

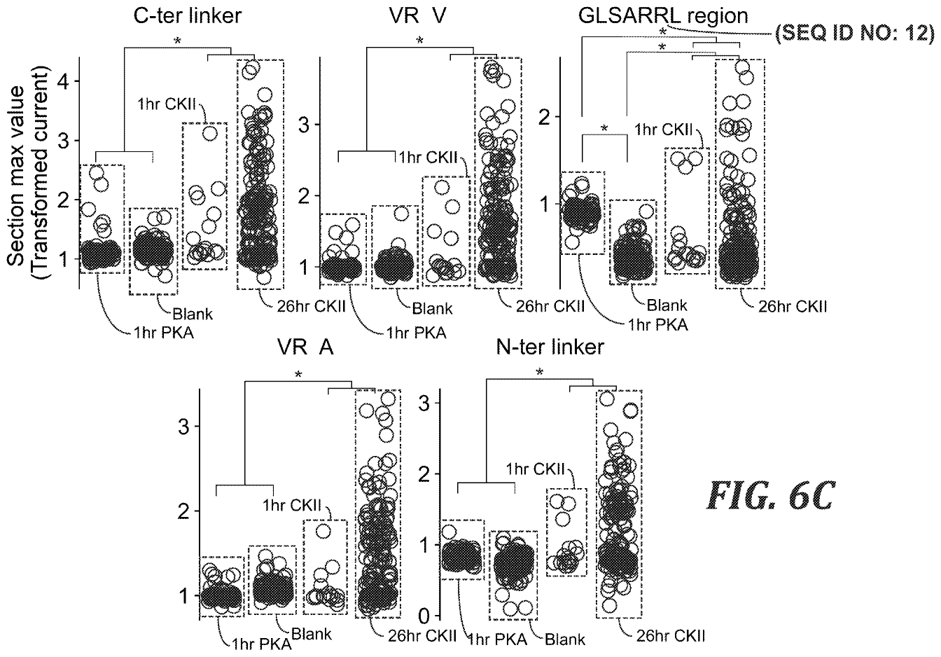

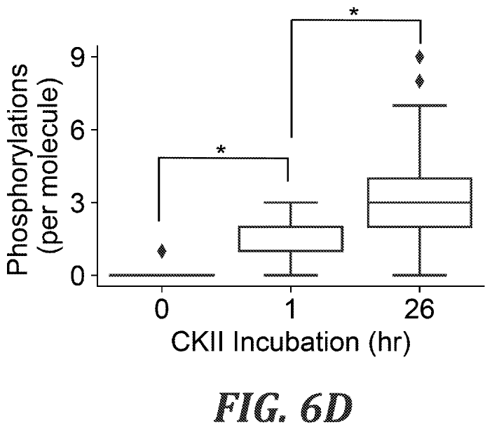

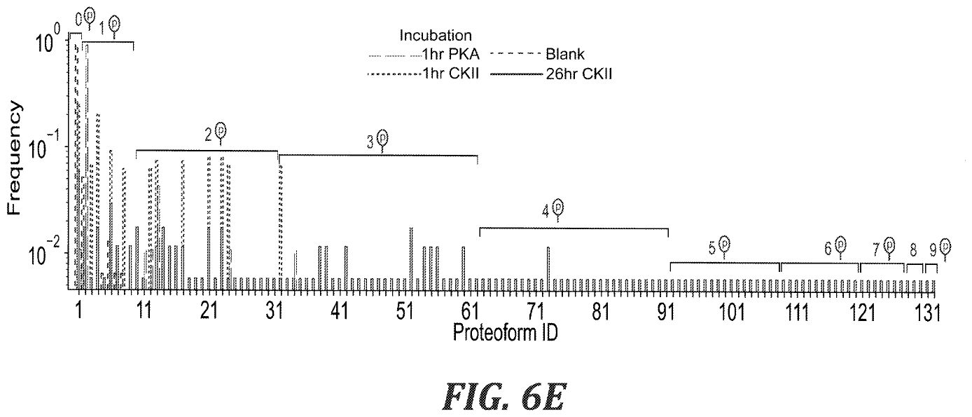

Phosphorylation detection: Each section (C-terminal linker, VR V, VR GLSARRL, VR A, and N-terminal linker) was extracted with YY-segmentation. For each section, the transformed current was aligned to the model of all possible phosphorylation states. The number of phosphorylations in each section was determined by the number of phosphorylations in the best-matching (i.e., lowest DTW distance) phosphorylation state model to the actual trace. When describing the signal increase in VR GLSARRL (SEQ ID NO: 11) due to PKA (Results, FIG. 6C), only the portion of the section up to the

$${\frac{n}{3}}^{th}$$

index, where n is the length of the YY-segmented VR GLSARRL (SEQ ID NO: 11), was used because that is where PKA causes signal increase, as seen in FIG. 6B.

### Null Hypothesis Tests

All PERMANOVA tests were performed on the DTW distance matrix of signals using scikit-bio and 10⁶ permutations, unless a Bonferroni correction was used, in which case n×10⁶ permutations were used, where n is the number of comparisons performed. Kruskal-Wallis, T, and Mann Whitney U tests were performed with scipy. Reported p-values were multiplied by n if we noted that we used a Bonerroni correction. All tests performed were two-sided, unless marked otherwise. P-values were considered significant if P<0.05.

### Results

First, the protein substrate is threaded into the nanopore via electrophoretic force. Then, ClpX is added to the first compartment solution to steadily pull the substrate protein back out of the pore (second compartment to first compartment) (FIG. 1A).

A protein was first synthesized to evaluate this method, which comprised an unstructured, negatively-charged N-terminal sequence of 42 amino acids rich in glycine, serine, and aspartic acid (polyGSD) to facilitate electrophoretic capture in the pore, attached to a stably folded domain (Smt3). This was followed by a short, positively charged sequence (RGS repeat) and a ClpX-binding ssrA tag (AANDENYALAA, (SEQ ID NO: 5) at the C-terminal end (referred to as protein P1). The combination of the RGS and the folded domain was included in the design to inhibit complete translocation of the protein through the pore, thereby preserving the ssrA tag's accessibility in the first compartment. After introducing P1 into a MinION R9.4.1 flow cell, which incorporates a CsgG pore variant (Oxford Nanopore Technologies), and applying a voltage of −180 mV, current blockades associated with the capture of the negatively charged protein tail was observed within the pores. To ascertain whether ClpX could be used to extract the captured protein from the nanopore, a buffer solution containing ClpX and ATP was introduced into the flow cell. Under these conditions, events were observed in which the deep ionic current blockade, characteristic of capture of the substrate protein in the nanopore, returned to the open channel state in a near stepwise manner sometime after ClpX addition (FIG. 1B). It was also determined that these events were ATP-dependent and occurred at a slower rate in the presence of ATPYS, an ATP analog that is more difficult for ClpX to hydrolyze. These results are consistent with the model that ClpX was binding to the ssrA tag and translocating the captured protein out of the nanopore in the C-to-N terminal directionality.

If this was true, it was reasoned that mutations in the tail domain of the protein would induce alterations in the ionic current states observed during ClpX-mediated translocation of the protein through the nanopore. In order to test this, three new proteins (P2, P3, and P4) were synthesized, each containing several tyrosine (Y) mutations at distinct positions of the polyGSD sequence. To directly compare the signal profiles of the four protein sequences, ensemble ionic current traces were created for each of these proteins, shown in FIG. 1C. This revealed that the major differences across the translocation signals corresponded with the respective positions of the tyrosine mutations along the different protein strands. Moreover, comparing all-vs-all signal DTW distances revealed that the sets of translocation signals generated by each unique protein sequence formed distinct clusters, differentiating them from every other protein. This was statistically supported by a p_{PERMANOVA} <1×10⁻⁶ for each comparison, after applying a Bonferroni correction.

### Determining the Ability to Resolve Individual ClpX Steps and Single-Amino Acid Substitutions

Having established the ClpX approach, the sensitivity of this method to single-amino acids was investigated as a first step towards developing a long-read protein analysis method. To do this, protein constructs that included five repeating sequence blocks were designed, each containing 59 amino acids. These blocks were built with a base sequence of glycine, serine, aspartic acid, and glutamic acid (FIG. 2A). A unique amino acid mutation was introduced at the central position within each block and demarcated the blocks with a double tyrosine mutation at each end. This design was used to minimize potential overlapping signal contributions from the mutations by ensuring sufficient spacing to prevent concurrent pore occupancy by multiple mutations. This hypothesis was grounded on prior observations indicating that around 20 amino acids can occupy the CsgG sensing region when in a stretched conformation. These strategically designed protein constructs were termed Proteins for Amino acid Sequencing Through Optimized Regions (PASTORs). A total of eight different PASTOR variants were synthesized, each containing a different sequence of mutations. The PASTOR design allowed us to analyze up to five different mutations in a single nanopore read, and the total set of eight pastors allowed us to interrogate each of the 20 amino acids in two different PASTOR sequence contexts.

ClpX-mediated analysis of the PASTOR proteins manifested elongated ionic current traces containing repetitive patterns that resulted from the seven YY mutations, seen as seven repeated dips in the signal preceding return to open channel (PASTOR-HDKER is shown in FIG. 2B). Between these dips, distinctive and reproducible variations in the ionic current signals were observed, corresponding with the variable amino acid mutation within each block. Utilizing the consistent, substantial effect of the YY mutations, the current signals were segregated into regions termed ‘YY dips’ and ‘variable regions’ (VRs) and used these patterns to scale our signals (Methods).

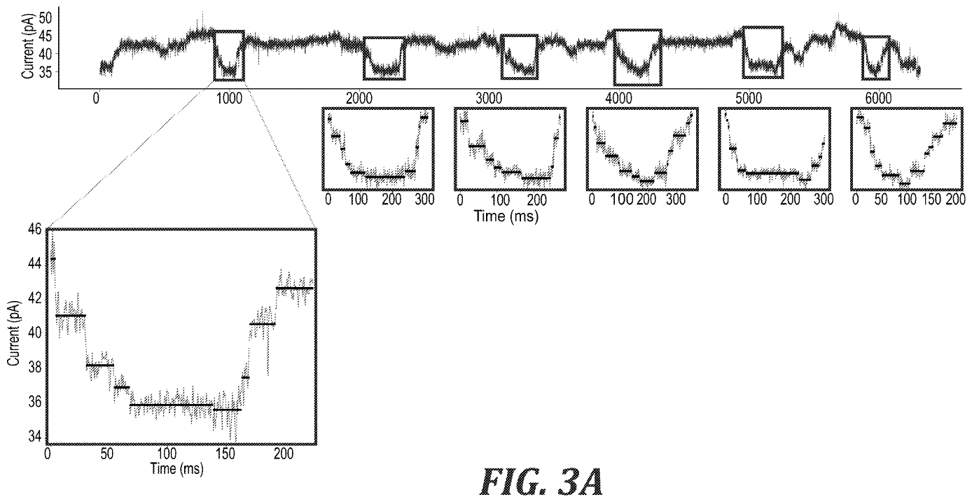

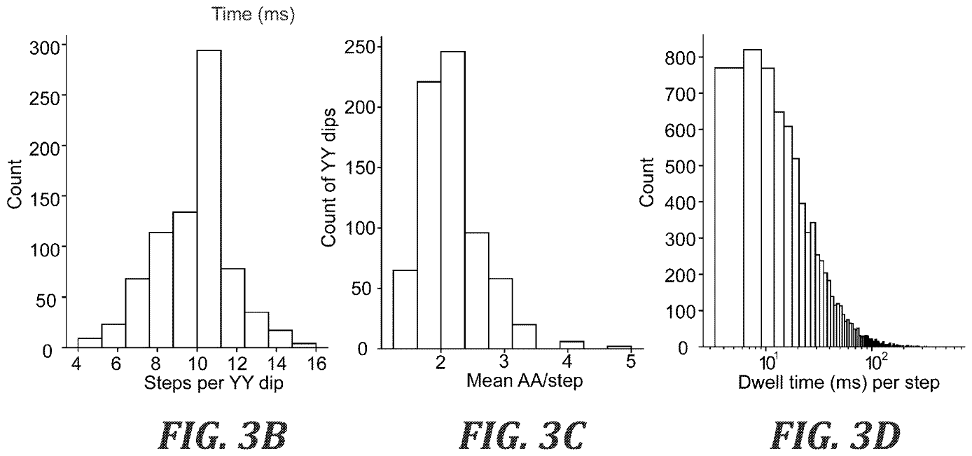

A close examination of the YY dip signals showed rapid, stepwise changes in the current level, which it was reasoned are due to single ClpX substrate translocation steps (FIG. 3A). To gain a deeper understanding of the translocation kinetics during protein reading, it would be useful to establish the ClpX step size and rate as it pulls the protein through the pore. Although single-molecule tweezer experiments have suggested that ClpX moves in 1 nm steps, translating to approximately 5-8 amino acids per step, this hypothesis conflicts with structural studies. Such studies indicate that related protein remodeling enzymes, structurally similar to ClpX, interact with two residues during their catalytic cycle, implying a fundamental step size of two amino acids.

To ascertain ClpX's step size in our experiments, we analyzed a total of 776 YY dip regions using a Bayesian-based segmentation algorithm, filtering out regions with backstepping or excessive noise (see Methods). By dividing the number of residues contributing to the YY dips (mean=19.1, and standard deviation, SD=2.71) by the number of steps identified per YY dip (mean=9.72, SD=1.94) (FIG. 3B), it was determined that ClpX translocates an average of ~1.96 residues per step (SD=0.25) (FIG. 3C). This was confirmed by a secondary t-test based segmentation, used in a different study of ClpX stepping behavior, yielding a similar mean of ~1.89 residues per step (SD=0.28). Additionally, the dwell time of each step, capturing the duration ClpX pauses between pulling events, had a mean of 28.6 ms (SD=32.3 ms) (FIG. 3D). These results are in strong agreement with the two amino acid step size hypothesized from the structural studies and suggest that the tweezer experiments lacked the spatiotemporal resolution to resolve individual ClpX steps.

After establishing ClpX's two-residue stepping behavior, the focus shifted to the variable regions (VRs) to explore the ionic signatures of individual amino acid mutations. The present analysis revealed that in VRs with a neutral amino acid mutation, there was a negative correlation between the ionic current levels and the volume of the amino acid (FIGS. 2C and 2D). This observation supports a volume exclusion model, where larger amino acids block more current than their smaller counterparts. Interestingly, the VRs containing positively charged residues (K/R) decreased the current level below the baseline sequence, while negatively charged residues increased it, diverging from the volume exclusion model. This effect was more substantial for negatively charged residues than for positively charged ones. One possible explanation for this could be that the negatively charged residues resist translocation to the negatively charged first compartment, causing the protein strand to stretch and consequently decrease the total volume of protein in the pore. Conversely, a positively charged residue would be attracted to the first compartment and could introduce upstream kinks in the protein strand, adding additional protein volume in the pore. The impact on signal levels could also be attributed to variations in solvation states and the mobility of ions near the charged amino acids. Collectively, these results showed that this method was sensitive to single amino acid residues.

### Detection of a Non-Enzymatic Post-Translational Modification

The present analyses also revealed substantial read-to-read signal variability within asparagine (N) and cysteine (C) VRs (FIG. 2E). The pronounced signal variability associated with cysteine (FIG. 2F) is likely due to its capacity to assume a spectrum of oxidation states. Signal traces for asparagine VRs, however, consistently displayed one of two distinct signal levels. The majority of signal events presented a minor dip, consistent with the volume exclusion model (FIGS. 2C and 2G). Conversely, around 10% of reads, under standard experimental conditions, exhibited a high signal peak, mirroring those of the acidic amino acid VRs aspartate and glutamate. It was hypothesized that these deviant reads might arise from post-translational asparagine deamidation, a process which transforms the asparagine sidechain to aspartate or isoaspartate. This conversion is recognized as a common PTM and is speculated to operate as an important biological timing mechanism in-vivo. To investigate this reasoning, the PASTOR-VGDNY protein was exposed to buffer conditions that promote the deamidation of asparagine to (iso) aspartate (Methods). Following protein incubation in this buffer, nanopore analysis of asparagine VRs revealed that approximately 96% now exhibited signal traits similar to aspartate (FIGS. 2G-2I). This increase in aspartate-like signals post-incubation supports our hypothesis that the atypical reads in the asparagine VRs originate from post-translational deamidation.

Modeling Nanopore Signals Directly from Amino Acid Sequence

Considering the relationship between the volume and charge of individual amino acids and their impact on nanopore signals, a biophysical model designed to simulate nanopore signals from a protein's amino acid sequence directly was developed. This model, building on Cardozo et al.'s findings, determines a summation of the volume and charge of amino acids within a moving 20-residue window, applying a centrally positioned negative parabolic weight. To quantitatively evaluate the congruency between our model and actual ionic current traces, their average distance post-DTW for a given sequence were calculated, denoted as S, post-normalizing both the model and experimental current traces. This value was designated as DTWDS and compared it to the distribution of DTW distances between the actual ionic current traces and the model of randomly composed sequences of amino acids derived from the distribution of amino acids in S, referred to as R. The average DTWDS for PASTORs ranked in the top 0.3% of the best matches within R. This evaluation affirms that the signal agreement is not due to artifacts from DTW alignment, thereby reinforcing the assertion that our model has the capacity to simulate these current traces accurately within these sequence contexts.

### Building an Aminocaller for Single-Molecule Sequencing

Sequencing PASTOR VRs would mark an important step in nanopore protein sequencing development. In addition, sequencing synthetic protein constructs such as PASTORs could serve diverse technological applications, including protein barcoding. This was addressed by initially training machine learning models to identify the single mutation present within a VR. This process consisted of filtering and scaling each of the raw signal traces, followed by segmentation of the VR signal regions (FIG. 4A). To featurize the VR signals, we used a combination of manually curated features and DTW-distance features (Methods). The latter helped to quantify the pairwise similarity of all the VR reads in the training set. Next several classical and deep machine learning models were explored and found that random forests most frequently achieved the highest accuracy. All classification analyses were then performed with a hyperparameter-tuned random forest evaluated on a fixed held-out test set, unless otherwise specified. Amino acid discrimination was explored across all pairs of amino acids (FIG. 4B). The highest accuracy classifications were achieved for pairs of amino acids with dissimilar volumes or when one was negatively charged, for example, tyrosine vs aspartate, which exhibited 100% discrimination accuracy. Some pairs of amino acids, such as leucine and isoleucine, proved to be more challenging due to their inherent physicochemical similarities. Amino acids with high variance signals, like cysteine, were also more difficult to distinguish from others. The models were then trained to classify amongst particular sets of three amino acids (for example G, Y, and D), in which the model achieved 95% single-read accuracy. Expanding this to five-way classifications (for example G, V, W, R, and D), the model maintained high performance, achieving an accuracy rate of 86%. In the most challenging task of all 20-way amino acid classification, our top-performing model substantially outperformed a dummy classifier, obtaining an accuracy of around 28%, compared to the latter's 5.5% (FIG. 4C). When top-N accuracy measurements were considered, our model attained 67% accuracy for top-5 and 81% for top-8 accuracy in the 20-way classification task (FIG. 4C). Lastly, the potential benefits of a higher salt buffer (400 mM KCl) on sequencing performance were investigated, hypothesizing that increased ionic strength might amplify signal distinctions and thus enhance classification accuracy. However, the models' performance was consistent across both standard and elevated salt conditions.

Building upon these results, the classifiers were integrated downstream of the PASTOR segmenter to develop an end-to-end PASTOR “aminocaller.” A set of PASTOR reads were aminocalled from the classification test set (FIGS. 4D and 4E). Overall sequencing accuracy per read averaged about 62% and 42% for the HDKER sequence, and roughly 51% and 21% for the AVLIM sequence, using five-way and 20-way classification models respectively. These results expand the capabilities of nanopore protein reading, moving beyond discriminating among a limited set of amino acids to sequencing individual amino acids across a protein strand, albeit in a synthetic sequence background.

### Rereading Single Protein Strands Using an Unfoldase Slip Sequence

After developing the aminocaller, we aimed to enhance the accuracy of our single-molecule sequencing approach by developing a method to reread single protein molecules. A multi-read strategy would facilitate the generation of consensus sequencing data at the individual molecule level. It has been indicated that ClpX may have difficulty gripping particular polypeptide sequences, such as polyproline, on which the ClpXP complex showed slow degradation rates. This prompted the hypothesis that incorporating a “slippery” amino acid sequence near the N-terminal of a PASTOR would induce ClpX to momentarily lose hold of the strand (FIG. 5A). Consequently, the substrate protein would be free to rethread into the pore via electrophoresis. Rethreading would cease and enzyme-mediated translocation would resume once ClpX regains its grip on the substrate. To test this strategy, a new PASTOR (PASTOR-reread) was constructed with two important sequence features: 1) a proline-rich “slip” sequence repeat (EPPPP) 5 (SEQ ID NO: 1) positioned near the N-terminal, and 2) VRs separated by an increasing number of tyrosine residues, ranging from two to five. It was reasoned that these distinctive tyrosine repeat lengths would aid in characterizing rereading events, given the distinct current levels produced by each repeat length. Reinforcing this hypothesis, our sequence-to-signal model predicted that this unique PASTOR-reread sequence design would generate a distinctive stair-step-like signal in the Yn-dips, enabling estimation the slip distance. Indeed, nanopore signals produced by PASTOR-reread generally exhibited repeated signal patterns that closely aligned with the model's prediction before returning to open channel (FIG. 5B). By using the tyrosine repeat regions as a measure of slip length, slipping distances were observed that were most often either within short ranges (50-100 amino acids) or extending across the entire PASTOR unstructured region (>300 amino acids), accounting for roughly 40% and 30% of all rereads, respectively (FIG. 5C). The distribution of these slipping distances remained relatively unchanged regardless of ClpX concentration. However, the frequency of slips, whether partial or full, did vary with changes in ClpX concentration (FIGS. 5D and 5E). At a concentration of 1000 nM, a median of 5.5 reads per PASTOR-reread molecule was observed, whereas at a lower concentration of 8 nM, we observed 22.5 reads per PASTOR-reread molecule (FIG. 5D). These results suggest that higher ClpX concentrations may lead to multiple ClpX complexes loading onto a single PASTOR strand. This increased loading could potentially reduce the frequency of back-slipping, as downstream ClpX complexes might serve as a backstop for the upstream ClpX encountering the slip sequence region. While prior studies have documented ClpX back-slipping during unsuccessful protein unfolding, these findings show direct evidence of purely sequence-dependent ClpX back-slipping. This suggests that this technique could also enable new single-molecule biophysical studies on protein-processive motors, paralleling nanopore research on DNA-processive motors.

The potential of single-molecule rereading in enhancing sequencing accuracy was investigated. While training our models similarly as before, the testing approach was modified. Instead of directly comparing the predicted classification to the actual label, test VRs with the same amino acid into clusters of size n (representing n reads, or n−1 rereads) were organized. Within each group, the class prediction with the highest average confidence was selected to represent the entire group. Using this approach, the accuracy for the all 20-way amino acid classification task improved from 28% to 61% (compared to a 5% random baseline) with 10 rereads (FIG. 5F). Likewise, the accuracy for a 7-way classification task improved from 66% to 99% (against a 14% random baseline). This method of estimating the effects of rereading offers promising indications of its potential to increase single-molecule sequencing accuracies. It was also postulated that the use of reread data in practice could offer even greater accuracy improvements. This is especially likely in a scenario where all the reads are considered in summation by the aminocaller models, as opposed to merely leveraging individual prediction confidences.

### Estimating Protein Barcode Sequencing Space and Accuracy

Having determined the capacity for high-accuracy sequencing through PASTOR rereading and the ability to design PASTOR proteins with customizable VR sequences, the PASTOR VR sequence space with varying constraints was simulated, with a view toward applications in protein barcoding. Based on the accuracy rates of our models (FIG. 5F), the number of distinct barcodes that could be generated at a given accuracy level was computed. This calculation considered varying degrees of rereading and two different VR segment numbers per protein barcode (5 and 10 VRs). A protein containing L VRs, where each VR could possess one of a possible amino acid mutations (where a=size of the mutation alphabet), would represent L×log₂(a) bits and could create a total of a^{L} unique barcodes. However, by devoting some of those bits to linear error-correcting codes for parity checks, we can enhance the decoding accuracy, although it results in a reduced barcode space. For example, our findings indicate that with 10 VRs and 10 rereads, it is feasible to generate libraries of over 1 million or 1 billion unique PASTOR barcodes that are decodable with a single-molecule accuracy >95% or >81%, respectively (FIG. 5G).

### Monitoring and Mapping Enzymatic Phosphorylation Along Single Protein Strands

Demonstrating the ability to detect and map phosphorylations across long protein strands would represent an important step toward developing a technology capable of identifying distinct full-length proteoforms. In pursuit of this, two serine/threonine protein kinases with distinct recognition motifs were analyzed: protein kinase A (PKA), which recognizes the canonical motif RRXS, and casein kinase II (CKII), which targets the sequence SXXD/E. To see if effective characterization of the differential activity of these two kinases using our nanopore reading approach was possible, a new substrate, PASTOR-phos (FIG. 6A), was designed. In this design, PKA's substrate peptide LRRASLG (“kemptide”) was inserted into one of the variable regions, making it specific for the kinase's recognition. For CKII interrogation in PASTOR-phos, the original PASTORs' 29 amino acid linker sequences were maintained, which inherently contain a CKII motif (GGSSDSSGSGSSESGSESSGSGSSDSSGG (SEQ ID NO: 10)), while reducing the total number of VRs.

After incubating PASTOR-phos with PKA for one hour, nanopore analysis was conducted. This analysis showed that a substantial increase in ionic current was observed in 91 out of 92 reads (98.9%) within the kemptide VR compared to the baseline. Specifically, the maximum transformed current levels rose to 0.9 (SD=0.09) from a baseline of 0.39 (SD=0.12), suggesting successful detection of phosphorylation in this sequence region (FIGS. 6B and 6C). This increase in ionic current is consistent with expectations for the negatively charged phosphoserine, which carries a charge of −2, to enhance ionic flow. Conversely, 361 out of the 368 non-kemptide VRs and linker sequences (98.1%) showed no substantial signal changes. These results are consistent with PKA activity being specific to the RRXS motif.

When the same substrate (PASTOR-phos) was treated with CKII for one hour, high read-to-read variability was observed manifested by large increases in current levels that were found to be concentrated in the eight linker sequences containing the CKII phosphorylation motif (FIG. 6A). The VRs and linker sequences incubated in CKII had a maximum peak transformed current of 1.4 (SD=0.66) and 387 out of 935 (41%) showed a significant increase; this is compared to a maximum peak transformed current of 0.93 (SD=0.20) in the baseline (no kinase incubation) VRs and linker reads, with only 5 out of 775 (0.6%) showing such increase (FIG. 6C). These observations support the method's ability to discern site-specific phosphorylation events, this instance showcasing specificity to CKII. Interestingly, a small portion of linkers had signal increases much higher than other ones, suggesting that they were being phosphorylated to a higher extent (FIGS. 6A and 6C). Upon analysis of the phosphorylated linker sequence, it was observed that phosphorylation at the initial motif induces the formation of a secondary CKII motif, SXXpS (GGSSDSSGSGSSEpSGSESSGSGSSDSSGG (SEQ ID NO: 10)), which has been described in the literature. It was hypothesized that linkers with significantly higher signal levels were phosphorylated at both serine positions. To test this, it was reasoned that extending the CKII incubation time with the substrate should increase the frequency of both single and, consequently, double phosphorylation events. Supporting this hypothesis, data from PASTOR-phos after a 26-hour incubation revealed increased occurrences of both putative single and double phosphorylations within the linker sequences (FIGS. 6C and 6D). Specifically, when comparing 1-hour to 26-hour incubations, the average number of identified single and double linker phosphorylations per molecule rose from 0.7 (SD=0.9) to 1.7 (SD=1.3) and from 0.06 (SD=0.3) to 0.6 (SD=0.9), respectively.

Given the PASTOR-phos sequence's abundance of potential CKII phosphorylation sites, numerous combinatorially unique proteoforms are possible (a total of 13,122). To map reads to these various modified forms, phosphoserine was integrated into our sequence-to-signal model (Methods). This approach allowed alignment of nanopore traces with the predicted sequence-to-signal profiles for each phosphorylation state across all variable regions (VRs) and linkers. Consequently, we identified and quantitatively assessed over one hundred distinct full-length proteoforms of PASTOR-phos, across reads obtained from the baseline, PKA, and CKII experiments (FIG. 6E). For example, the 26-hour CKII incubation resulted in single molecules containing as many as 9 phosphorylated residues. This highlights the capability of our nanopore reading approach to detect and map phosphorylation sites, as well as to observe the varied activities of different kinases on single protein molecules.

### Processive Reading of Folded Protein Domains

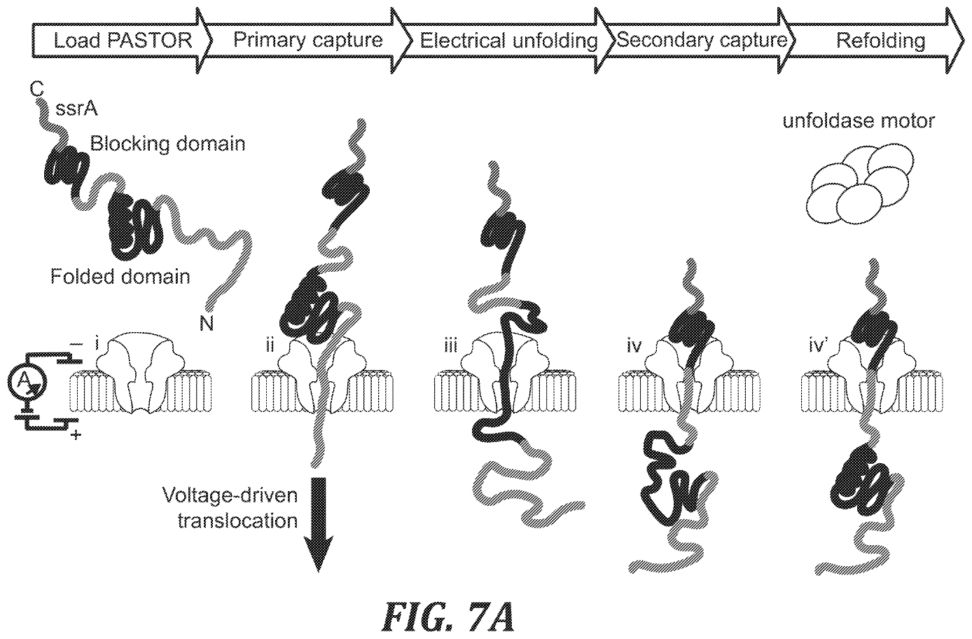

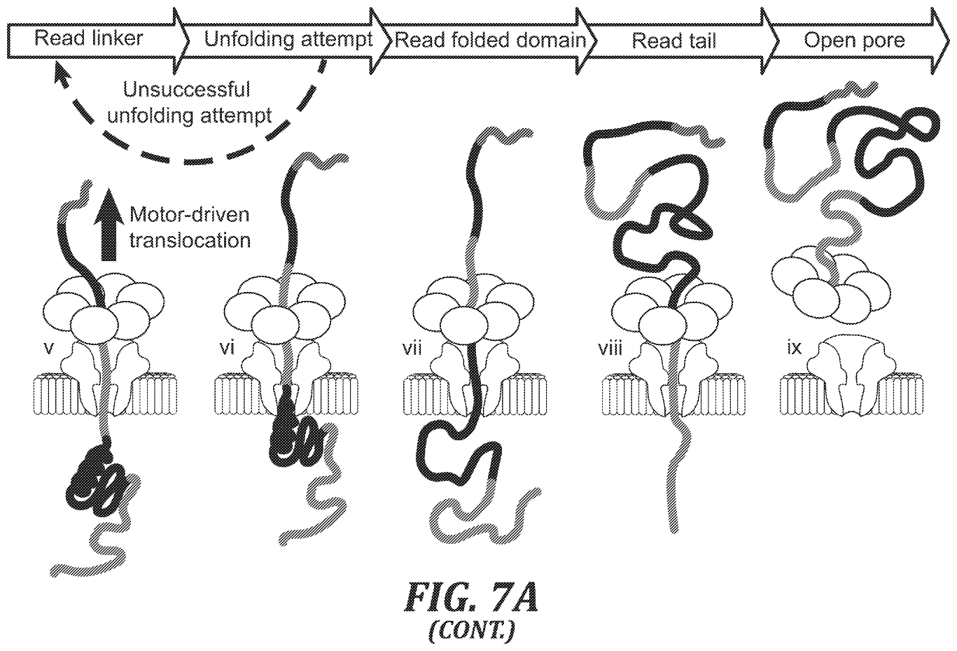

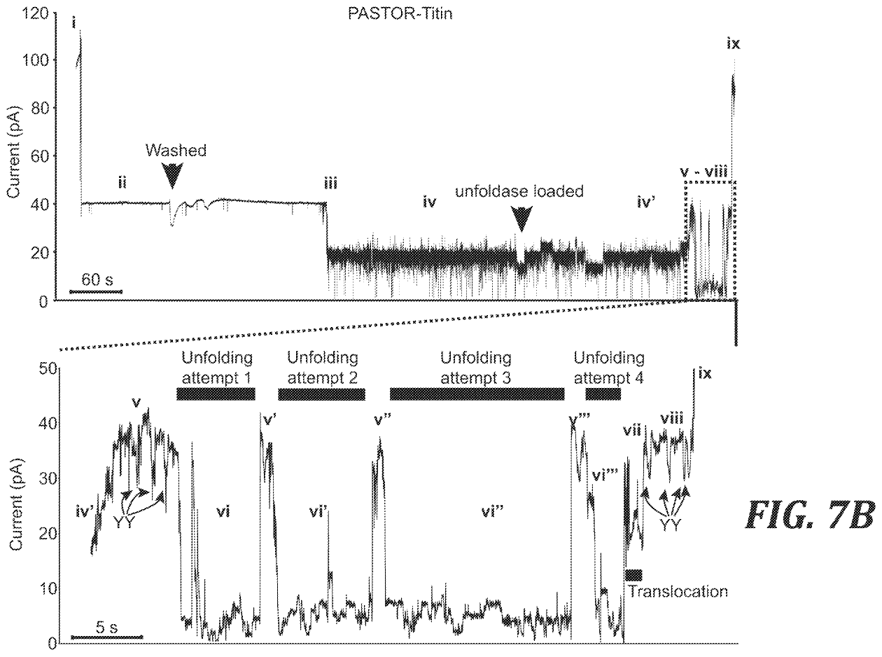

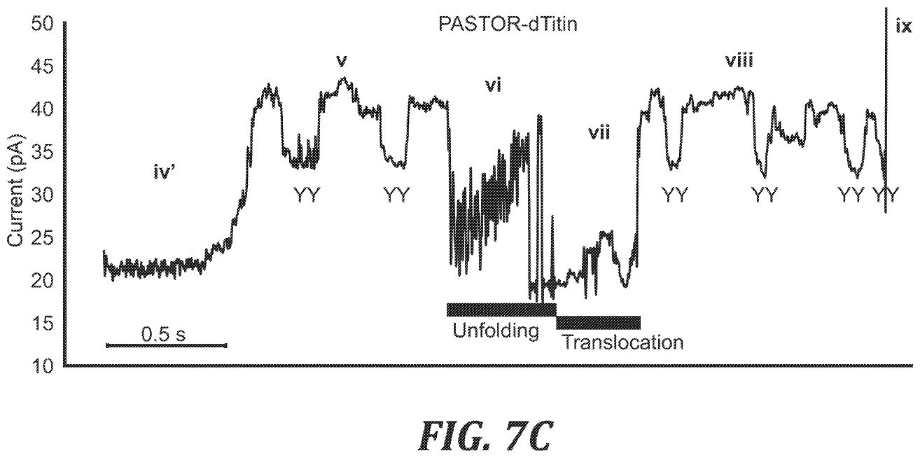

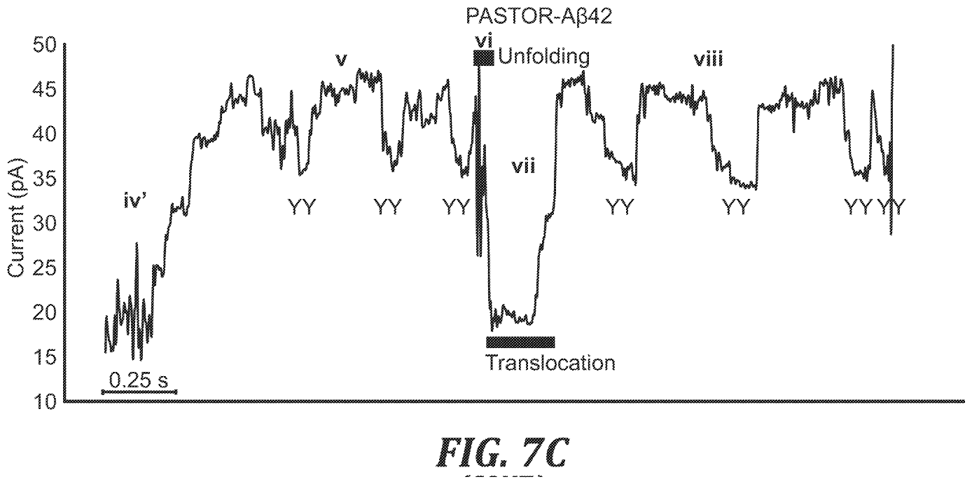

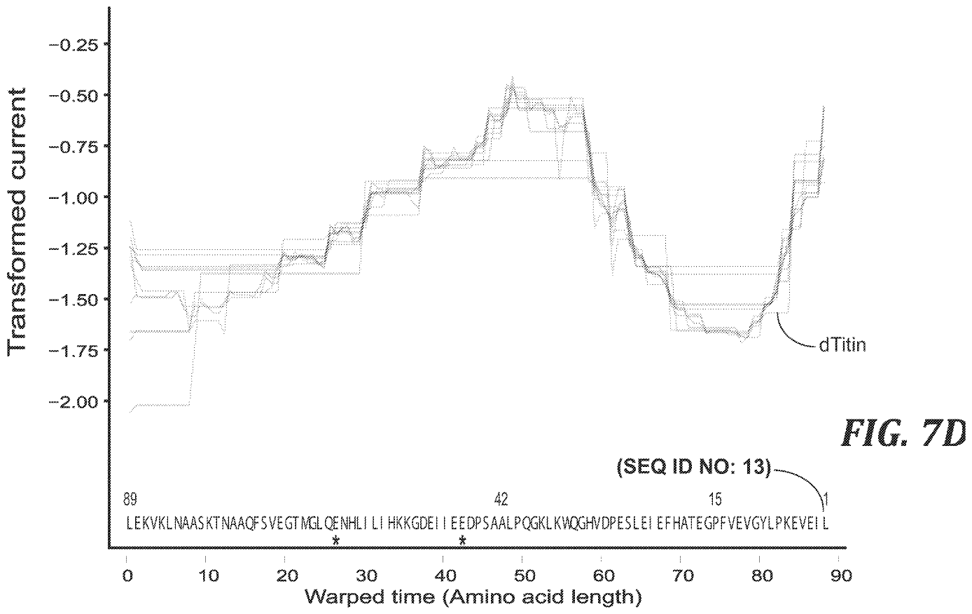

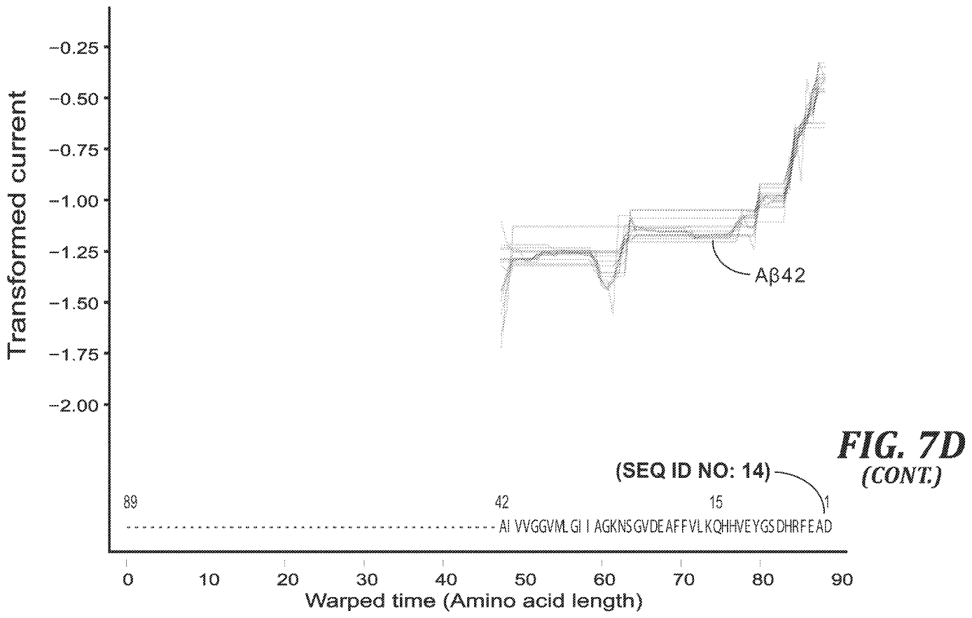

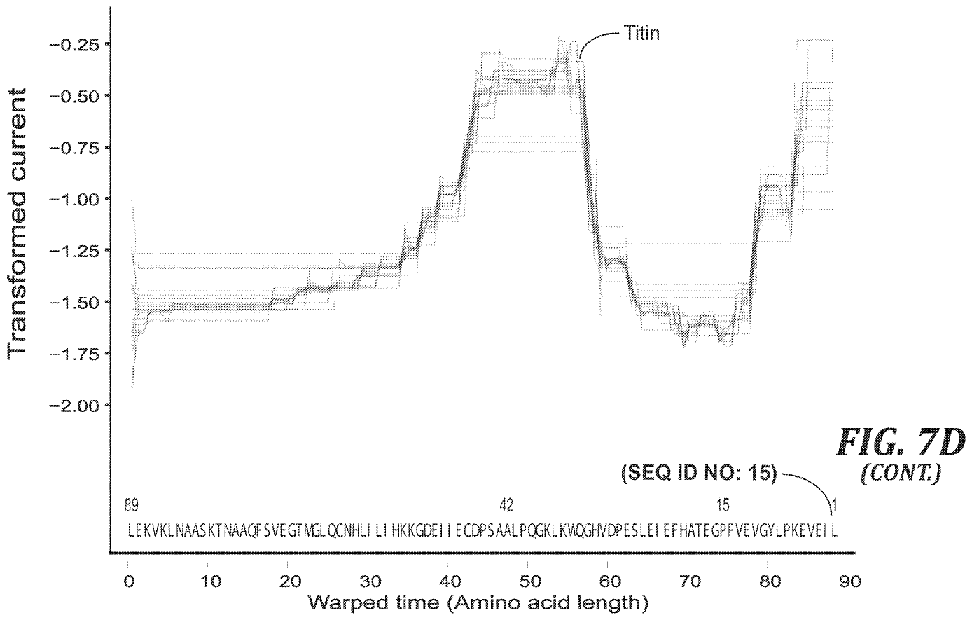

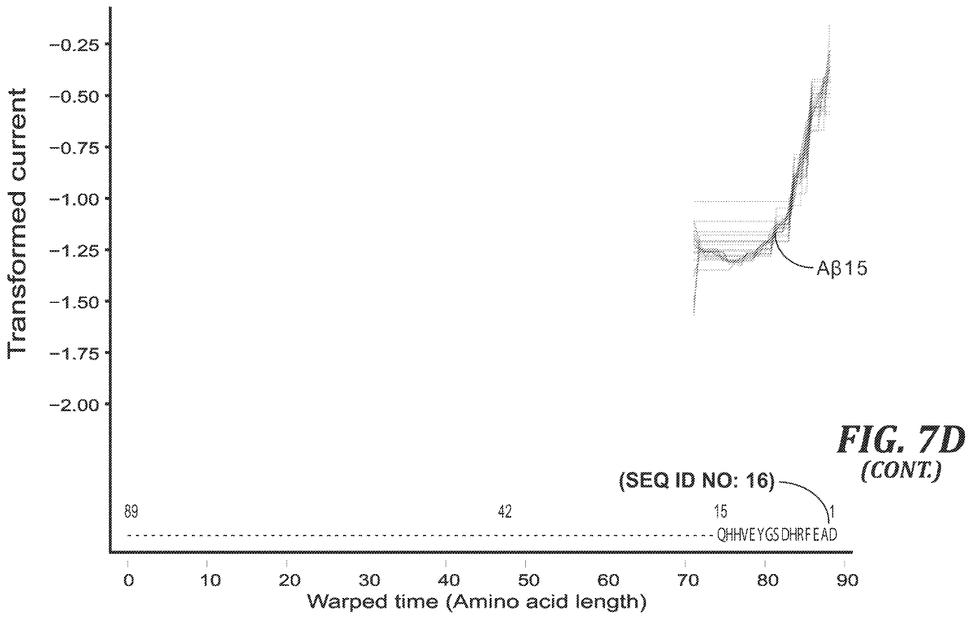

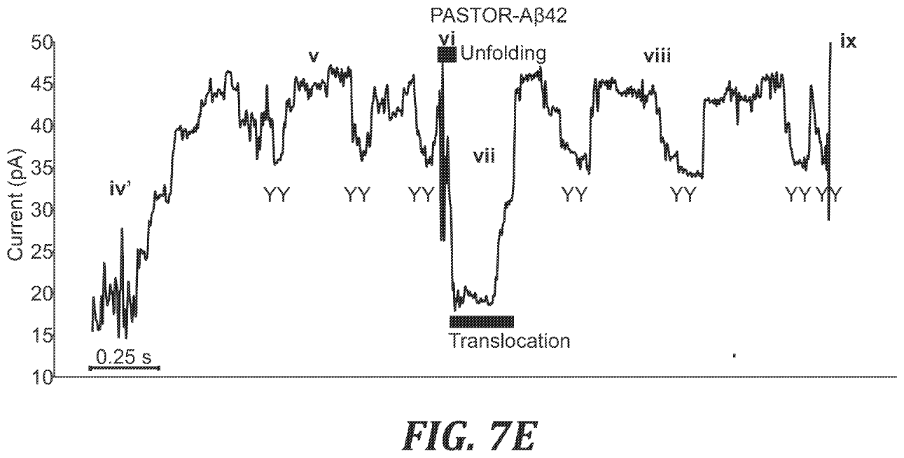

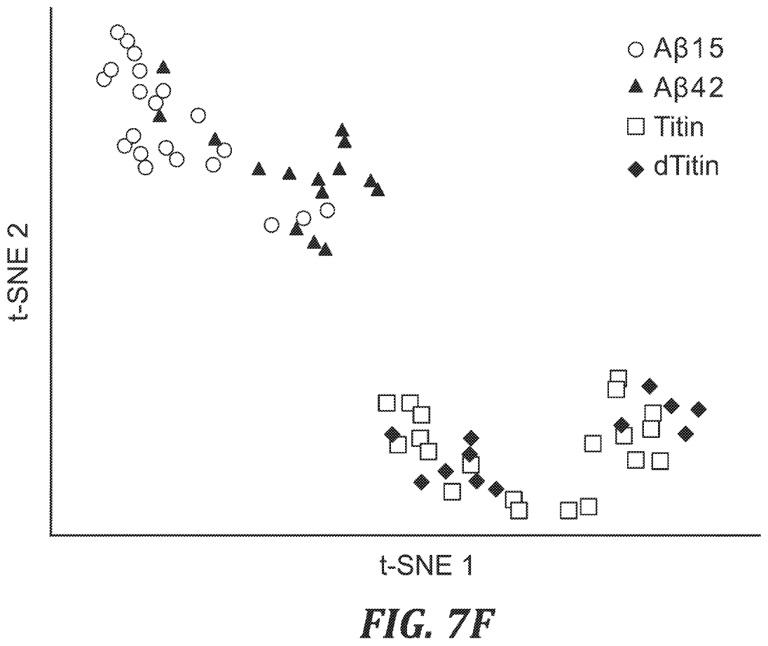

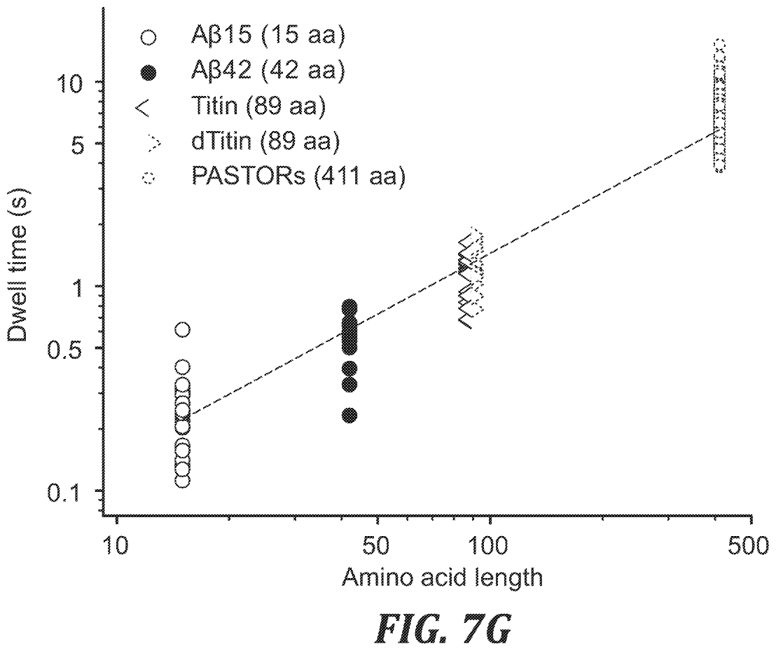

Progressing beyond synthetic, unstructured sequences, the effectiveness of unfoldase-based method was evaluated on protein sequences that contain a folded domain. For this purpose, a PASTOR protein with the titin I27_{V15P} domain, which consists of 89 amino acids arranged into 8 β-strands, was analyzed inserting into the third VR position (PASTOR-Titin). Unlike unstructured proteins, nanopore traces of PASTOR-Titin yielded an initial two-step electrophoretic nanopore capture state, indicating that the folded titin domain was first captured atop the nanopore at state ii and then electrically unfolded at transition state iii to produce the typical PASTOR capture signal at state iv manifested by the Smt3 domain atop the pore (FIGS. 7A and 7B). Following the addition of ClpX to the first compartment, a translocation signal corresponding to the leading VRs and YY regions, which are tethered to the C-terminus of the titin domain (state v) was observed. Subsequently, a distinct and deep blockade state was observed, which was interpreted as ClpX attempting to unfold the titin domain (state vi), which presumably refolds in the second compartment after initial translocation. This deep blockade state often reverted back to the previous state, indicating an unsuccessful ClpX unfolding attempt and suggesting that ClpX slipped back on the protein strand. Following a successful unfolding attempt, putative translocation of the titin domain (state vii) was observed. After translocation of the titin domain, characteristic signal features corresponding to the latter VRs and YY regions (state viii) before transitioning back to an open pore state (state ix) (FIG. 7B) were observed.

To affirm this understanding of the unfolding and translocation states, we performed experiments using a variant of titin I27 (PASTOR-dTitin) with a destabilized tertiary structure, introduced through double-point mutations (C47E, C63E) on two buried cysteines. Comparing PASTOR-dTitin with PASTOR-Titin allowed us to explore the effect of titin's tertiary structure on the resulting current signals. PASTOR-dTitin generated traces that bore resemblance to those from PASTOR-Titin but with two notable differences (FIG. 7C). First, PASTOR-dTitin displayed unique signal features at the putative unfolding state vi, indicating structural disparities between the two variants. Second, states v-vi were observed only once in PASTOR-dTitin before the presumptive translocation state vii, in contrast to PASTOR-Titin where they were typically observed multiple times, leading to a substantial difference between the distribution of PASTOR-Titin and PASTOR-dTitin unfolding times. These differences can be attributed to PASTOR-Titin's more stable, unfolding-resistant titin domain compared to that of PASTOR-dTitin. In PASTOR-Titin, repeated observations of states v and vi, which were not present in PASTOR-dTitin, support the conclusion that they result from unsuccessful unfolding attempts and ClpX back-slipping events, triggered by the stable titin domain. Also, despite their dissimilar structural stabilities, PASTOR-Titin and PASTOR-dTitin demonstrated similar signals during the putative translocation state vii (FIG. 7D). This similarity reflects their nearly identical primary amino acid sequences. The observation of similar signals at the proposed translocation state vii between PASTOR-Titin and PASTOR-dTitin, despite their differences in structural stability, underscores the role of the primary amino acid sequence in this process. It suggests that the primary sequence is the major determinant of the translocation signal through the nanopore, while structural variations exert a more significant influence on the preceding unfolding state.

PASTOR constructs with the amyloid beta protein 1-42 (PASTOR-Aβ42) and its shorter derivative 1-15 (PASTOR-Aβ15), which have distinct amino acid sequences and lengths from the titin domain were next tested. It was reasoned that Aβ42 and Aβ15 would generate brief unfolding states as they are partially but not fully structured in their monomeric forms. As expected, upon nanopore analysis they yielded ionic current traces similar to PASTOR-dTitin overall but with distinct features in unfolding state vi (FIG. 7E). Further, comparing their putative translocation states (state vii) using DTW distances, it was observed that the signals generated by PASTOR-Aβ42 and PASTOR-Aβ15 share similarities, while being distinct from signals generated by PASTOR-Titin and PASTOR-dTitin (FIGS. 7D and 7F), reflective of the translocation state being dependent on protein primary sequence. Overall, the dwell times of these different states also corresponded well with their respective sequence lengths across all the PASTOR proteins, suggesting a translocation rate of ~63 amino acids/second (average dwell time of ~16 milliseconds/amino acid) (FIG. 7G). This is close to previous estimates of ClpX translocation speed, and the observation that the rate of ClpX-mediated protein translocation is relatively constant regardless of protein sequence. Altogether, these results provide evidence that the unfolding state vi and translocation state vii independently record structural and sequence information, respectively, showing the potential of this technique to enable multimodal protein analysis on a single platform.

Finally, the predictive model was assessed using these proteins. As the current model does not factor in the signal features linked with unfolding, the signal segment following the unfolding state until the completion of the translocation (i.e. states vii-viii) was analyzed. Using the same comparison technique as previously implemented for the PASTOR protein models, it was found that the average DTWDS for the PASTORS containing folded domains ranked in the top 0.04% of the best matches within R. This evaluation is evidence that our model can adequately simulate these current traces in the specified sequence contexts.

### Discussion

Provided herein is a new approach for single-molecule reading of long protein strands using nanopores and an unfoldase motor protein. This method achieves single-amino acid sensitivity and demonstrates the capability to reread and sequence amino acid substitutions across long protein strands. This opens new opportunities for the advancement of protein barcoding technology, as we project the ability to design large libraries of synthetic peptide sequences (>1B) that can be decoded with high accuracy. Moreover, method was applied to detect and map the activities of distinct kinases, achieving site-specific detection of enzymatic post-translational modifications (PTMs) along extended protein sequences and the relative quantification of >100 putative proteoforms of a single protein substrate. Although PTMs are crucial for proteoform diversity, their quantitative analysis, especially in single-molecule contexts, is difficult due to resolution and throughput constraints of current technologies such as mass spectrometry. The kinase assays not only demonstrate the capability to analyze PTMs such as phosphorylation on whole proteins but also suggest a pathway towards integrating barcoding with PTM analysis for potential multiplexing.

Stemming from this method, exploration into ClpX stepping activity revealed a deeper understanding of its operational kinetics. It is established herein that ClpX translocates proteins through the nanopore in a stepwise manner, closely aligning with structural studies that suggest a fundamental step size of two amino acids. Provided herein is a biophysical model capable of simulating nanopore signals that are generated as individual protein sequences are pulled through the nanopore by the unfoldase. This result enables a “lookup table” approach reminiscent of mass spectrometry, facilitating full-length, single-molecule protein identification and fingerprinting. This strategy would entail creating a database comprising simulated nanopore signals associated with various target protein sequences (including PTMs), potentially encompassing an entire proteome, and could be generated computationally given a robust sequence-to-signal predictor. The raw data derived from actual nanopore readings could then be compared against this database to identify the best protein match based on raw signal alignment, for example by utilizing DTW. This offers an attractive alternative to sequence-based matching, which is a complex task due to the challenge of accurately mapping ionic current to specific amino acid sequences, given the large number of amino acids contributing to the nanopore signal at any one time.

Additionally, it is demonstrated herein a full-length reading of folded protein domains, an important result as we move towards reading of natural protein molecules. In the present system, electrophoretic (FIG. 7A; state iii) and ClpX-mediated (FIG. 7A; state vii) protein unfolding is important to achieving full-length folded domain analysis. It is likely some protein domains may exhibit a greater resistance to unfolding than the substrates explored in this study. In such cases, additional strategies could be used to facilitate unfolding, such as the use of denaturants and electroosmotic flow. The present methodology can include synthetic N- and C-terminal sequences, which can be appended using existing termini-specific chemical conjugation techniques. This approach mirrors the adapter ligation step that is used for high-throughput DNA sequencing methods.

In conclusion, provides methods useful in full-length protein identification, capable of achieving the highest level of proteoform resolution. Additionally, the methods described herein provide immediate advancements, particularly in the context of protein barcoding and PTM monitoring applications.

### DEFINITIONS

### Nucleic Acid:

A nucleic acid is a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxymethylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

The five-carbon sugar to which the nucleobases are attached can vary depending on the type of nucleic acid. For example, the sugar is deoxyribose in DNA and is ribose in RNA. In some instances herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyrobo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, a ribonucleotide (RNA) polymer. The nucleic acids can also be or comprise a PNA polymer, or a combination of any of the polymer types described herein (e.g., contain residues with different sugars).

### Peptide

As used herein, the term “peptide” refers to natural biological or artificially manufactured short chains of amino acid monomers linked by peptide (amide) bonds. As used herein, a peptide has at least 2 amino acid repeating units.

### Polypeptide/Protein

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

### Protein

As used herein, the term “protein” refers to any of various naturally occurring substances that consist of amino-acid residues joined by peptide bonds, contain the elements carbon, hydrogen, nitrogen, oxygen, usually sulfur, and occasionally other elements (such as phosphorus or iron), and include many essential biological compounds (such as enzymes, hormones, or antibodies).

### EMBODIMENTS

The present disclosure includes the following embodiments.

1. A method of analyzing a polypeptide, the method comprising:
providing, into a first compartment of a nanopore sequencing device, one or more polypeptides comprising:
a polyanion sequence;
a folded domain stably folded at neutral pH and/or a positively charged sequence;
an unfoldase slip sequence; and
an analyte sequence,
applying an electrophoretic and/or electroosmotic force between the first compartment and a second compartment of the nanopore sequencing device to translocate partially the one or more polypeptides from the first compartment to the second compartment through a nanopore of the nanopore sequencing device;
translocating, with an unfoldase enzyme, the one or more polypeptides partially through the nanopore from the second compartment to the first compartment; and
measuring a current corresponding to moving, with the unfoldase enzyme, the one or more polypeptides from the second compartment to the first compartment.

2. The method of Embodiment 1, wherein the unfoldase slip sequence is configured to cause the one or more proteins to translocate from the first compartment into the second compartment.

3. The method of Embodiment 1, wherein a current pattern from the measured current corresponds to translocating, with the unfoldase, the one or more polypeptides at least partially from the second compartment to the first compartment a plurality of times.

4. The method of Embodiment 3, further comprising combining portions of the current pattern corresponding to translocating the one or more polypeptides from the second compartment to the first compartment to provide a combined current pattern.

5. The method of Embodiment 4, further comparing a portion of the combined current pattern corresponding to the analyte sequence to a reference current pattern obtained using a reference polypeptide measured under analogous conditions.

6. The method of Embodiment 5, further comprising generating an analyte sequence call based on comparing the portion of the combined current pattern corresponding to the analyte sequence to reference current pattern the reference polypeptide analyte measured under analogous conditions.

7. The method according to any of Embodiments 1-7, wherein the folded domain is disposed in a C-terminal direction relative to the analyte sequence.

8. The method according to any of Embodiments 1-7, wherein the unfoldase slip sequence is disposed in an N-terminal direction relative to the analyte sequence.

9. The method according to any of Embodiments 1-8, wherein the positively charged sequence is disposed in a C-terminal direction relative to the analyte sequence.

10. The method according to any of Embodiments 1-9, wherein the positively charged sequence is disposed in an N-terminal direction relative to the analyte sequence.

11. The method according to any of Embodiments 1-10, wherein the polyanion sequence is disposed in a C-terminal direction relative to the analyte sequence.

12. The method according to any of Embodiments 1-11, wherein the unfoldase slip sequence comprises polyproline sequences.

13. The method according to any of Embodiments 1-12, wherein the unfoldase slip sequence comprises one or more of a sequence according to SEQ ID NO. 1.

14. The method according to any of Embodiments 1-13, wherein the unfoldase slip sequence comprises one or more of a sequence according to SEQ ID NO. 2.

15. The method according to any of Embodiments 1-14, wherein the unfoldase comprises an amino acid sequence at least 90% or 95% identical to the amino acid sequence of SEQ ID NO: 3.

16. The method according to any of Embodiments 1-15, wherein the nanopore comprises an amino acid sequence at least 90% or 95% identical to the amino acid sequence of SEQ ID NO: 4.

17. The method according to any of Embodiments 1-16, wherein the polyanion sequence comprises a plurality of amino acid residues that are anionic under neutral pH.

18. The method according to any of Embodiments 1-17, wherein the polyanion sequence comprises at least 10 amino acid residues, at least 20 amino acid residues, at least 30 amino acid residues, at least 40 amino acid residues, or at least 50 amino acid residues.

19. The method according to any of Embodiments 1-18, wherein the polyanion sequence is configured to facilitate voltage-mediated capture of the one or more polypeptides in the nanopore.

20. The method according to any of Embodiments 1-19, wherein the polyanion sequence comprises amino acids selected from the group consisting of aspartic acid and glutamic acid.

21. The method according to any of Embodiments 1-20, wherein the folded domain is configured to prevent complete translocation of the one or more polypeptides through the nanopore.

22. The method according to any of Embodiments 1-21, wherein the folded domain comprises a tertiary structure larger than a vestibule of the nanopore.

23. The method according to any of Embodiments 1-22, wherein positively charged sequence prevents complete translocation of the one or more polypeptide through the nanopore.

24. The method according to any of Embodiments 1-23, wherein positively charged sequence comprises amino acid residues selected from the group consisting of arginine, histidine, and lysine.

25. The method according to any of Embodiments 1-24, wherein the unfoldase is configured to unfold the folded domain.

26. The method according to any of Embodiments 1-25, wherein the one or more polypeptides further comprise an ssrA tag.

27. The method of Embodiment 26, wherein the ssRA comprises a sequence according to SEQ ID NO: 5.

28. The method according to any of Embodiments 26 or 27, wherein the ssRA is disposed in a C-terminal direction of the analyte sequence.

29. The method according to any of Embodiments 1-28, further comprising modifying one or more sample polypeptides to comprise the polyanion sequence; the folded domain, the positively charged sequence; and the unfoldase slip sequence to provide the one or more polypeptides.

30. A kit comprising:
a nanopore sequencing device comprising:
a first compartment;
a second compartment;
a nanopore disposed in an insulating membrane fluidically separating the first compartment from the second compartment;
an unfoldase disposed in either the first compartment or the second compartment; and
one or more unfoldase slip sequences.

31. The kit of Embodiment 30, further comprising reagents configured to functionalize a sample polypeptide with the one or more unfoldase slip sequences.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the present disclosure have been described herein for purposes of illustration, various modifications may be made without deviating from the spirit and scope of the present disclosure. Accordingly, the invention is not limited except as by the claims.

The embodiments of the invention in which an exclusive property or privilege is claimed are defined as follows:

1. A method of analyzing a polypeptide, the method comprising:
providing, into a first compartment of a nanopore sequencing device, one or more polypeptides comprising:
a polyanion sequence;
a folded domain stably folded at neutral pH and/or a positively charged sequence;
an unfoldase slip sequence; and
an analyte sequence,
applying an electrophoretic and/or electroosmotic force between the first compartment and a second compartment of the nanopore sequencing device to translocate partially the one or more polypeptides from the first compartment to the second compartment through a nanopore of the nanopore sequencing device;
translocating, with an unfoldase enzyme, the one or more polypeptides partially through the nanopore from the second compartment to the first compartment; and
measuring a current corresponding to moving, with the unfoldase enzyme, the one or more polypeptides from the second compartment to the first compartment.

2. The method of claim 1, wherein the unfoldase slip sequence is configured to cause the one or more proteins to translocate from the first compartment into the second compartment.

3. The method of claim 1, wherein a current pattern from the measured current corresponds to translocating, with the unfoldase, the one or more polypeptides at least partially from the second compartment to the first compartment a plurality of times.

4. The method of claim 3, further comprising combining portions of the current pattern corresponding to translocating the one or more polypeptides from the second compartment to the first compartment to provide a combined current pattern.

5. The method of claim 4, further comparing a portion of the combined current pattern corresponding to the analyte sequence to a reference current pattern obtained using a reference polypeptide measured under analogous conditions.

6. The method of claim 5, further comprising generating an analyte sequence call based on comparing the portion of the combined current pattern corresponding to the analyte sequence to reference current pattern the reference polypeptide analyte measured under analogous conditions.

7. The method of claim 1, wherein the folded domain is disposed in a C-terminal direction relative to the analyte sequence.

8. The method of claim 1, wherein the unfoldase slip sequence is disposed in an N-terminal direction relative to the analyte sequence.

9. The method of claim 1, wherein the positively charged sequence is disposed in a C-terminal direction relative to the analyte sequence.

10. The method of claim 1, wherein the positively charged sequence is disposed in an N-terminal direction relative to the analyte sequence.

11. The method of claim 1, wherein the polyanion sequence is disposed in a C-terminal direction relative to the analyte sequence.

12. The method of claim 1, wherein the unfoldase slip sequence comprises polyproline sequences.

13. The method of claim 1, wherein the unfoldase slip sequence comprises one or more of a sequence according to SEQ ID NO: 1.

14. The method of claim 1, wherein the unfoldase slip sequence comprises one or more of a sequence according to SEQ ID NO: 2.

15. The method of claim 1, wherein the unfoldase comprises an amino acid sequence at least 90% or 95% identical to the amino acid sequence of SEQ ID NO: 3.

16. The method of claim 1, wherein the nanopore comprises an amino acid sequence at least 90% or 95% identical to the amino acid sequence of SEQ ID NO: 4.

17. The method of claim 1, wherein the polyanion sequence comprises a plurality of amino acid residues that are anionic under neutral pH.

18. The method of claim 1, wherein the polyanion sequence comprises at least 10 amino acid residues, at least 20 amino acid residues, at least 30 amino acid residues, at least 40 amino acid residues, or at least 50 amino acid residues.

19. The method of claim 1, wherein the polyanion sequence is configured to facilitate voltage-mediated capture of the one or more polypeptides in the nanopore.

20. The method of claim 1, wherein the polyanion sequence comprises amino acids selected from the group consisting of aspartic acid and glutamic acid.

21. The method of claim 1, wherein the folded domain is configured to prevent complete translocation of the one or more polypeptides through the nanopore.

22. The method of claim 1, wherein the folded domain comprises a tertiary structure larger than a vestibule of the nanopore.

23. The method of claim 1, wherein positively charged sequence prevents complete translocation of the one or more polypeptide through the nanopore.

24. The method of claim 1, wherein positively charged sequence comprises amino acid residues selected from the group consisting of arginine, histidine, and lysine.

25. The method of claim 1, wherein the unfoldase is configured to unfold the folded domain.

26. The method of claim 1, wherein the one or more polypeptides further comprise an ssrA.

27. The method of claim 26, wherein the ssRA tag comprises a sequence according to SEQ ID NO: 5.

28. The method of claim 26, wherein the ssRA tag is disposed in a C-terminal direction of the analyte sequence.

29. The method of claim 1, further comprising modifying one or more sample polypeptides to comprise the polyanion sequence; the folded domain, the positively charged sequence; and the unfoldase slip sequence to provide the one or more polypeptides.

30. A kit comprising:
a nanopore sequencing device comprising:
a first compartment;
a second compartment;
a nanopore disposed in an insulating membrane fluidically separating the first compartment from the second compartment;
an unfoldase disposed in either the first compartment or the second compartment; and
one or more unfoldase slip sequences.

31. The kit of claim 30, further comprising reagents configured to functionalize a sample polypeptide with the one or more unfoldase slip sequences.


SEQUENCE LISTING (16 sequences)

SEQ ID NO: 1 - protein, 5 aa, synthetic construct
EPPPP

SEQ ID NO: 2 - protein, 5 aa, synthetic construct
KPPPP

SEQ ID NO: 3-4 - skipped (2 entries)

SEQ ID NO: 5 - protein, 11 aa, synthetic construct
AANDENYALAA

SEQ ID NO: 6 - protein, 39 aa, synthetic construct
SDDGDGGEGGDDGGGDSGDGDSDGDGSGGGSDGGSSGGG

SEQ ID NO: 7 - protein, 39 aa, synthetic construct
SDDYDYYEGGDDGYGDSGDGDSDGDGSGGGSDGGSSGGG

SEQ ID NO: 8 - protein, 39 aa, synthetic construct
SDDYDYYEGGDDGYGDSGDGDSDGDGSYYYSDGGSSGGG

SEQ ID NO: 9 - protein, 39 aa, synthetic construct
SDDYDYYEGGDDGYGDSGDGDSDGDGSGGGSDGGSSYYY

SEQ ID NO: 10 - protein, 59 aa, synthetic construct
GGSSDSSGSGSSESGSESSGSGSSDSSGGXGGSSDSSGSGSSESGSESSGSGSSDSSGG

SEQ ID NO: 11 - protein, 29 aa, synthetic construct
GGSSDSSGSGSSESGSESSGSGSSDSSGG

SEQ ID NO: 12 - protein, 7 aa, synthetic construct
GLSARRL

SEQ ID NO: 13 - protein, 89 aa, synthetic construct
LEKVKLNAASKTNAAQFSVEGTMGLQENHLILIHKKGDEIIEEDPSAALPQGKLKWQGHVDPESLEIEFHATEGPFVEVGYLPKEVEIL

SEQ ID NO: 14 - protein, 42 aa, synthetic construct
AIVVGGVMLGIIAGKNSGVDEAFFVLKQHHVEYGSDHRFEAD

SEQ ID NO: 15 - protein, 89 aa, synthetic construct
LEKVKLNAASKTNAAQFSVEGTMGLQCNHLILIHKKGDEIIECDPSAALPQGKLKWQGHVDPESLEIEFHATEGPFVEVGYLPKEVEIL

SEQ ID NO: 16 - protein, 15 aa, synthetic construct
QHHVEYGSDHRFEAD